(19) United States
(12) Patent Application Publication
(10) Pub. No.: US 2016/0235889 A1
(43) Pub. Date: Aug. 18, 2016

(54) SILK/PLATELET COMPOSITION AND USE THEREOF

(71) Applicant: TUFTS UNIVERSITY, Medford, MA (US)

(72) Inventors: Isabella Pallotta, Medford, MA (US); Jonathan Kluge, Southborough, MA (US); Alessandra Balduini, Pavia (IT); David L. Kaplan, Concord, MA (US)

(73) Assignee: Tufts University, Medford, MA (US)

(21) Appl. No.: 15/025,049

(22) PCT Filed: Sep. 25, 2014
(86) PCT No.: PCT/US2014/057541

§ 371 Date: Mar. 25, 2016

Related U.S. Application Data
(60) Provisional application No. 61/883,726, filed on Sep. 27, 2013.

(51) Int. Cl. A61L 27/36; A61K 35/19; A61K 8/98; A61K 38/48; A61K 8/66; A61K 9/00; A61K 9/06; A61K 8/04; A61L 27/52; A61L 27/54; A61K 8/64; A61K 38/18; A61L 27/58; A61Q 19/08; A61Q 7/00; A61K 35/64

(52) U.S. Cl. CPC A61L 27/3604; A61L 27/3616; A61K 35/64; A61K 35/19; A61K 8/987; A61K 8/983; A61K 38/4833; A61K 8/66; A61K 9/0019; A61K 9/0014; A61K 9/06; A61K 8/042; A61L 27/52; A61L 27/54; A61K 8/64; A61K 38/18; A61L 27/58; A61Q 19/08; A61Q 7/00; A61L 2300/414; A61L 2300/412; A61L 2300/604; A61L 2300/45; A61L 2300/418; A61K 2800/5922; A61L 2300/62

(57) ABSTRACT

The present disclosure relates generally to silk fibroin and unlysed platelet compositions. The compositions are useful for a variety of medical uses such as wound healing or repair and soft tissue repair or augmentation.

### GOVERNMENT SUPPORT

This invention was made with government support under grant no. NIH P41 EB002520 awarded by the National Institute of Health. The government has certain rights in the invention.

### TECHNICAL FIELD

The present disclosure relates generally to compositions comprising silk and platelets and uses thereof.

### BACKGROUND

Platelets play a vital role in normal hemostasis and wound healing. After production by parent megakaryocytes (roughly 10¹¹ are produced daily by the body), they circulate throughout the blood stream in a latent state for an average of roughly 1 week prior to removal. In physiological conditions, circulating platelets are kept in an inactive state by prostacyclin and nitric oxide (NO) released by the endothelial cells that reside in the walls of blood vessels. During this time in circulation, they can become readily activated through a number of mechanisms, including surface protein interactions with insoluble tissue fragments, mechanical perturbations, or other chemical signals that result from tissue damage (Borzini et al., Isbt Science Series 2007, 2, 272-281). At sites of blood vessel injury, platelets adhere to the exposed subendothelium through interactions between von Willebrand factor, collagen and fibronectin and their receptors on the platelets, integrin α2β1, glycoprotein Ib-IX (GP Ib-IX) and integrin α5β1, respectively. Initially, Thrombin and adenosine diphosphate (ADP) lead platelets to change into an active conformation. In a positive feedback loop, activated platelets secrete additional ADP, platelet-derived growth factor, and fibrinogen from their storage granules, and thromboxane A2 (TXA2). ADP and TXA2 in turn cause circulating platelets to change shape and become activated. Glycoprotein IIb/IIIa receptors on the surface of activated platelets bind fibrinogen, leading to the formation of fibrinogen bridges between the platelets, resulting in platelet aggregation that, together with the simultaneous formation of a fibrin mesh generated by thrombin, lead to the formation of the platelet thrombus. Finally, the clot retraction leads to the formation of a stable thrombus.

For many years, the scientific community has recognized the role of platelets in tissue repair (Whitman et al., J. of Oral and Maxillofacial Surgery 1997, 55, 1294-1299; Marx et al., Oral Surgery Oral Medicine Oral Pathology Oral Radiology and Endodontics 1998, 85, 638; Anitua et al., Thrombosis and Haemostasis 2004, 91, 4-15; Aspenberg et al., Acta Orthop Scand 2004, 75, 93-99). More recently, the pharmaceutical community has begun to investigate implantable or injectable therapies that are enriched with supra-physiological levels of platelet growth factors (Kaux et al., Pathol Biol (Paris) 2011, 59, 157-160; Simonpieri et al., Current Pharmaceutical Biotechnology 2012, 13, 1231-1256). In particular, researchers have shown the benefits of one-time injections of so-called Platelet Rich Plasma (PRP)—the result of which, when in the presence of autologous thrombin and fibrinogen, is a fibrin hydrogel network containing activated platelets that can assemble in roughly 10-20 minutes. The fibrin hydrogel containing platelets has also been called a PRP-clot or Platelet Gel (PG), which are all synonymous with the activated contents of PRP. Platelet gels have been used for reconstructive oral and maxillofacial surgery (Whitman et al., J. of Oral and Maxillofacial Surgery 1997, 55, 1294-1299); achilles tendon repair (Aspenberg et al., Acta Orthop Scand 2004, 75, 93-99); anterior cruciate ligament repair (Murray et al., J. of Orthopaedic Research 2007, 25, 81-91); augmenting bone implants (Marx et al., Oral Surgery Oral Medicine Oral Pathology Oral Radiology and Endodontics 1998, 85, 638); augmenting dental implants (Whitman et al., J. of Oral and Maxillofacial Surgery 1997, 55, 1294-1299; Kontovazainitis et al., 2008, 28, 301-307; Nikolidakis et al., Clin. Oral Implants Res. 2008, 19, 207-213; Forni et al., Blood Transfus. 2013, 11, 102-107); bone and dental reconstruction (Marx et al., Oral Surgery Oral Medicine Oral Pathology Oral Radiology and Endodontics 1998, 85, 638); focal cartilage repair and microfracture augmentation (Milano et al., Osteoarthritis Cartilage 2010, 18, 971-980; Napolitano et al., Blood Transfus. 2012, 10, 72-77; Zhu et al., J. Tissue Eng. Regen. Med. 2012); and wound closure, diabetic foot ulcers (Driver et al., Ostomy Wound Manage. 2006, 52, 68-70, 72, 74 passim; Kim et al., Mol. Med. Report 2013, 7, 476-480; Lacci et al., Yale J. Biol. Med. 2010, 83, 1-9).

The PG can be exogenously applied to wound tissues, conferring benefits due to the released growth factors and the ability to localize the platelet concentrates in the site of injury because of gel formation. This method has been demonstrated to be more efficient than the use of recombinant growth factors (Bianco et al., Stem Cells 2001, 19, 180-192). The use of PG provides also a microenvironment for the sequential process of tissue regeneration involving migration, proliferation and differentiation of osteogenic and endothelial cells (Ogino et al., Oral Surgery Oral Medicine Oral Pathology Oral Radiology and Endodontics 2006, 101, 724-729; Cenni et al., J. Orthop Res. 2009, 27, 1493-1498). Because of the adherence nature of the gel, the PG can be injected alone or in combination with different bone substitutes avoiding unwanted migration of bone particles (Aghaloo et al., Int. J. Oral. Maxillofac. Implants 2004, 19, 59-65). The combination with bone allografts, as scaffolds, and bone marrow stromal cells is able to increase the efficacy of PG, as demonstrated in experimental animals and in clinical application (Dallari et al., J. Orthop. Res. 2006, 24, 877-888; Dallari et al., J. Bone Joint Surg. Am. 2007, 89, 2413-2420). Finally, the outcome of healing at the wound site can be influenced by the fibrin structure, in terms of thickness of the fibers, number of branch point, the porosity and the permeability of the clot (Laurens et al., J. Thromb. Haemost. 2006, 4, 932-939), suggesting that a method able to control the mechanical properties of the clot can lead to an improvement in wound healing.

Platelets regulate tissue remodeling by releasing the contents of their storage granules in the form of signaling molecules, including cytokines and chemokines that mediate the process of inflammation, as well as growth factors, which can have a more lasting effects on matrix synthesis and cellular proliferation (Anitua et al., Thrombosis and Haemostasis 2004, 91, 4-15). The most abundant of these growth factors include vascular endothelial growth factor (VEGF), transforming growth factor-β1 (TGF-β1), platelet-derived growth factor (PDGF), insulin-like growth factor-1 (IGF-1), and basic fibroblast growth factor (bFGF) (Anitua et al., Thrombosis and Haemostasis 2004, 91, 4-15).

Application of platelet rich plasma (PRP) in a liquid form to a wound site can be complicated by the loss of PRP in the surrounding site; therefore, PRP is more effective if used as a gel. However, the fundamental issues currently related to platelet gel administration are i) methods of application of gels, ii) the poor control of both concentration and timing of growth factor delivery, and iii) the mechanical robustness of the matrix upon administration, which generally limits applicability. Moreover, the contact between the platelet membrane and the fibrin clot with the surrounding tissues can cause immunogenic responses. Thus, there remains a need in the art for compositions and methods of their use that can overcome one or more of these problems.

### SUMMARY

Here the inventors present a new system in which an inert biomaterial, such as silk fibroin, is used to encapsulate the PG in order to avoid the contact between the wound site and the fibrin clot and to allow a controlled and sustained release of growth factors. During the extended lifetime of the silk encapsulation matrix, mechanical benefits can be conferred to the system during its slow kinetics of remodeling. Both the rate of degradation and mechanical properties of the silk-PG complex can be modulated in order to meet application-specific product specifications. In addition, the rate of growth factor delivery can be controlled by modulating the net charge of silk fibroin.

In one aspect, the disclosure provides a composition comprising silk fibroin and unlysed platelets. In some embodiments, the composition can be in the form of a gel or hydrogel. The composition disclosed herein can be used in a variety of medical fields: wound healing, orthopedics, dentistry, rheumatology, dermatology, and the like. The composition can be used for wound healing or repair, soft tissue repair or augmentation, fillers for tissue space, templates for tissue reconstruction or regeneration, scaffolds for cells in tissue engineering applications, as a vehicle/carrier for drug delivery, as a scaffold to mimic the extracellular matrices (ECM) of the body, and/or promote tissue regeneration. The composition can serve both as physical support and/or adhesive.

The composition disclosed herein can be used in various methods of medical treatments and diagnosis. For example, the compositions disclosed herein can be used in method for reconstructive oral and maxillofacial surgery, Achilles tendon repair, anterior cruciate ligament repair, augmenting bone implants, augmenting dental implants, bone and dental reconstruction, focal cartilage repair and microfracture augmentation, wound closure, diabetic foot ulcers, cosmetic surgery and medicine, hair loss treatment, wrinkle treatment and the like. The method generally comprises implanting, administering, or placing a composition as disclosed herein at the desired site of action. Accordingly, in another aspect, the disclosure provides a method of wound healing; soft tissue repair, augmentation, or reconstruction; hard tissue (musculoskeletal) repair, augmentation, or reconstruction; or filling a tissue located at or near a prosthetic implant. Generally, the method comprises implanting, administering, or placing a composition as disclosed herein at the desired sites, e.g. wound site.

### BRIEF DESCRIPTION OF THE DRAWINGS

FIG. 1 shows (A) Cumulative release of platelet growth factors in PBS. (i) Values were normalized to growth factor released by PG-PBS at the first time point and expressed as %. At each time point PG-Silk supernatant (PBS) were evaluated by ELISA. The insert in each graph represents the kinetic profile of PG-PBS. (ii) At the last time point PG-Silk samples were solubilized with urea 8M and the obtained solutions were evaluated by ELISA and normalized to PG-PBS at the first time point. (iii) recombinant VEGF (rVEGF) recovery after urea treatment. rVEGF was loaded to 2% silk solution and subjected to dialysis, or treatment with urea 8 M. rVEGF was loaded to 2% silk solution, sonicated to allow gelation, solubilized with urea 8 M and subjected to dialysis. Values were normalized to relative control (rVEGF in silk solution). (B) Contribution of silk fibroin ionomers towards release and recovery of growth factors from PG-Silk. (i) Silk solution was mixed or not with silk fibroin-poly-L-lysine and silk fibroin-poly-L-glutamic acid ionomers (0.1% w/v) prior to adding PRP in order to obtain PG-Silk. Growth factor release was measured from PBS overtime (21 days) or after gel solubilization with urea. Values were normalized to the relative growth factor released by PG after 1 hr of gelation. (ii) Swelling of PG-Silk and PG-Silk mixed with silk fibroin ionomers after incubation in PBS for 24 hrs.

FIG. 2 shows VEGF and pERK signaling in HUVEC proliferation induced by PG-Silk. (A) PBS supernatant collected from PG-Silk (25% v/v with control media) was used to inoculate HUVEC cells. The MTS assay was conducted after 3 days. * denotes statistically significant difference compared with the control medium (p<0.05). (B) Representative immunoblotting image showing increased extracellular signal-regulated kinase 1 and 2 (ERK1/2) phosphorylation in HUVEC cells cultured with PBS supernatant collected from PG-Silk, as compared to cells cultured with complete media. Actin was used as control of protein equal loading. (C) HUVEC cells were cultured in PBS supernatant collected from PG-Silk (25% v/v with control media) in presence of UO 126 or anti-VEGF neutralizing antibody. The MTS assay was conducted after 3 days (p<0.05). (D) Representative immunoblotting image showing a decreased ERK1/2 phosphorylation in HUVEC cells cultured 2 hrs with PBS supernatant collected from PG-Silk in presence of anti-VEGF. Actin was used as control of protein equal loading.

FIG. 3 shows the ability of silk to stabilize growth factors. (A) rVEGF stability in silk solution overtime. rVEGF was resuspended in PBS or in 2% silk solution and the concentration was assessed by Elisa overtime (16 days). (B) PG-Silk samples, upon gelation, were re-suspended in PBS or silk solution and TGFbeta1 release was measured after 7 days. (C) Growth factors in plasma, obtained from PG, were diluted 1:1 in PBS or 1:1 in 1% silk solution and maintained at 37° C., or room temperature, or 4° C. After 2 weeks the concentration of TGFbeta1 was assessed by Elisa.

FIG. 4 shows time-dependent rheology and compressive mechanics of silk and platelet gels. (A) Dynamic time-dependent rheological behavior of sonicated 10% (solid line), 4% (dashed line), and 2% w/v (dotted line) silk solutions compared to platelet gel (X marker) behavior over 6 hours of oscillatory shear. Not shown are the monotonically-increasing modulus values for the silk groups for up to 16 hrs, whereas the platelet gel group begins decreasing at ˜10 minutes of oscillatory shear. (B) Rheological behavior of sonicated 4% w/v silk solutions diluted 1:1 with platelet gel (+PG, solid line), Platelet Poor Plasma (+PPP, thick dotted line), or water as a control (fine dotted line). Platelet gels alone are also shown (X marker). (C) Re-plotted log-log data from (B) of the initial 100 minutes following gel initiation, showing the incidence of gel stiffening as indicated by the arrows. (D) Silk solutions were either autoclaved or sterile-filtered prior to mixing with either PG or PPP at a 1:1 dilution to generate the final silk concentration shown (therefore starting silk concentrations are 2× as listed, 10% was the maximum concentration that could be used from silk boiled for 30 minutes). Bar indicates significance between groups identified in post-hoc analysis of ANOVA (p<0.05).

FIG. 5 shows in vivo histology analysis of PGs and Silk-PGs. H&E staining of PG (A) and Silk-PG (B) after 12 hours from injection (i scale bar 200 μm, ii scale bar 100 μm), after 2 weeks from injection (iii scale bar 200 μm, iv scale bar 100 μm) and after 1 month from injection (v scale bar 200 μm, vi scale bar 100 μm). The arrows indicate the PG or Silk-PG constructs. The stars indicate aggregated platelets. The box indicates the area of infiltrated cells where high magnification pictures have been acquired. Images are representative of three independent experiments.

FIG. 6 shows in vivo immunohistochemistry analysis of PGs and Silk-PGs. (A) Ve-cadherin immunohistochemistry of PGs, (B) Silk-PG constructs, (C) CD31 immunohistochemistry of PGs, and (D) different Silk-PG constructs injected and analyzed after 12 hours (i), 2 weeks (ii), and 1 month (iii) (scale bar 100 m). Images are representative of three independent experiments.

FIG. 7 shows (A) The compressive mechanical properties of Silk-PG hydrogels comparing boil time and sterilization methods. Silk solutions used to generate the hydrogels were derived from cocoons boiled for either 30, 45, or 60 minutes (30 MB, 45 MB, or 60 MB) and each solution group was either autoclaved (“A”) or sterile-filtered (“F”) prior to mixing with PG. All silk solutions were concentrated 2× and then diluted 1:1 with PG to generate the final silk concentration shown. As indicated by *, 30 MB solution could not be filtered at 10w/v % (5% final conc.) or higher, whereas the cut-off for 45 MB and 60 MB filtration was 12w/v % (6% final conc.). 30 MB silk could not be autoclaved at 12 w/v %. Bar indicates significance between autoclaved or filtered treatment groups across boil times, identified in post-hoc analysis of two-way ANOVA (p<0.05). (B) PG reinforces silk gels independent of sterilization modality. Dynamic time-dependent rheological behavior of sonicated 4% w/v silk solutions diluted 1:1 with Platelet Gel (+PG, solid line), Platelet Rich Plasma (+PPP, thick dotted line), or water as a control (fine dotted line). Platelet gels alone are also shown (X marker, bottom left of each graph). Silk solutions are shown as untreated (Left), sterilized by 0.2 μm filter (Center), or 20 minute autoclave treatment (Right).

FIG. 8 shows in vivo histology analysis of PGs, silk gels and PG-Silk gels. Masson's Thricrome of different constructs injected and analyzed after 12 hours (i), 2 weeks (ii), and 1 month (iii) (scale bar 200 μm).

FIG. 9 shows in vivo histology analysis of silk gels. H&E staining of different constructs silk gels after 2 weeks from injection (i scale bar 200 μm, ii scale bar 100 μm) and after 1 month from injection (iii scale bar 200 μm, iv scale bar 100 μm).

FIG. 10 shows in vivo immunohistochemistry analysis of silk gels. (A) Ve-cadherin and (B) CD31 immunohistochemistry of different silk gel constructs injected and analyzed after 2 weeks (i), and 1 month (ii) (scale bar 100 μm).

### DETAILED DESCRIPTION

Inventors have discovered inter alia that compositions comprising silk fibroin and unlysed platelets provide a number of surprisingly unexpected properties and advantages. For example, the compositions disclosed herein provide one or more of the following: compositions can allow for substantially complete recovery of platelet growth factors; compositions can be available in multiple formulations for a large spectrum of clinical applications; compositions can be made allogenic to reduce or inhibit adverse immune reactions; compositions can be produced under mild conditions, which allows incorporating sensitive molecules/materials into the composition; compositions can be produced without any organic solvents; compositions can allow controlled release/function of platelet derived factors, mechanical properties can be optimized for the desired use; and additional agents can be added to the composition to optimize the release rate/kinetics of platelet derived factors.

Accordingly, in one aspect, the disclosure provides a composition comprising silk fibroin and unlysed platelets. The composition comprising silk fibroin and unlysed platelets is also referred to as silk/platelet composition or platelet/silk composition. Without limitations, the silk/platelet composition can be in any shape, form, or size. As used herein, the term “silk/platelet gel”, “silk/PG”, “PG-silk”, or any variants thereof, refers to a silk/platelet composition wherein the platelets are in a “platelet gel” form. The term “platelet gel” refers to the gelatin-like malleable product that results when platelet activators (such as thrombin and calcium) are added to platelet rich plasma. In some embodiments, the silk/platelet composition can be in the form of a gel or hydrogel. The term “hydrogel” is used herein to mean a silk-based material which exhibits the ability to retain a significant portion of water or other liquid within its structure without dissolution. Methods for preparing gels and hydrogels comprising silk fibroin are well known in the art. Exemplary methods for preparing silk fibroin gels and hydrogels include, but are not limited to, sonication, vortexing, pH titration, exposure to electric field, solvent immersion, water annealing, water vapor annealing, and the like. Exemplary methods for preparing silk fibroin gels and hydrogels are described in, for example, WO 2005/012606, WO 2008/150861, WO 2010/036992, and WO 2011/005381, content of all of which is incorporated herein by reference in its entirety. Gels formed by exposure to electric field are also referred to as e-gels herein. Methods for forming e-gels are described in, for example, US2011/0171239, content of which is incorporated herein by reference in its entirety.

In some embodiments, the composition can be in the form of a double-network gel or hydrogel. The double-network composition can comprises a first network and a second network, wherein the first network can comprise a platelet gel and the second network can comprises a silk fibroin gel. The first network can be semi-interpenetrated with the second network. In some embodiments, physical and/or mechanical properties of the double-network compositions can be controlled by gelation of PG and silk.

In some embodiments, the silk/platelet composition can be in the form of a sponge or foam. In some embodiments, the foam or sponge is a patterned foam or sponge, e.g., nanopatterned foam or sponge. Exemplary methods for preparing silk foams and sponges are described in, for example, WO 2004/000915, WO 2004/000255, and WO 2005/012606, content of all of which is incorporated herein by reference in its entirety.

Platelets, or thrombocytes, are small, disk shaped clear cell fragments (i.e. cells that do not have a nucleus), 2-3 μm in diameter, which are derived from fragmentation of precursor megakaryocytes. The average lifespan of a platelet is normally just 5 to 9 days. Platelets are a natural source of growth factors. They circulate in the blood of mammals and are involved in hemostasis, leading to the formation of blood clots.

The platelets in the silk/platelet composition can be activated. Activation of platelets is a process whereby platelets are converted from a quiescent, resting state to one in which platelets undergo a number of morphologic changes. These changes include changes in the shape of the platelets, accompanied by the formation of pseudopods, binding to membrane receptors, and secretion of small molecules and proteins (e.g., growth factors) and formation of platelet gel. Activated platelets release a multitude of factors including, but not limited to, cytokines (e.g., IL-1β, IL-6, TNF-α), chemokines (e.g., ENA-78 (CXCL5), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-β1, 2, and 3, and VEGF). Many of these growth factors have been shown to play a significant role in the repair and regeneration of connective tissues. For example, platelet-derived growth factor (PDGF), a potent chemotactic agent, and TGF beta, stimulates the deposition of extracellular matrix.

Without limitations, any agent, e.g., platelet activator, known in the art for activating platelets can be used for activating the platelets. Generally, the term “platelet activator” means a compound that is able to activate the release of platelet granule contents that promote the coagulation reactions. Exemplary platelet activators include, but are not limited to, thrombin, epinephrine, calcium salts, arachidonic acid, ADP, collagen, thromboxane A₂, ristocetin, TRAP (thrombin-receptor activation peptide; the peptide sequence is SFLLRN), PAF (platelet-activating factor), GPRP (the peptide gly-pro-arg-pro), serotonin, dopamine, collagen-related peptide, U-46619, coagulation pathway activators or agents (coagulation agent), batroxobin, and the like. In some embodiments, the platelets can be activated by thrombin and calcium gluconate, or calcium chloride, or by calcium salts without thrombin.

Accordingly, in some embodiments, the silk/platelet disclosed herein further comprises a platelet activator. Amount of the platelet activator in the silk/platelet composition can range from about 0.01% to about 50% (w/v).

The number of platelets in the silk/platelet composition can range from few hundred to a few million platelets per μL of the composition. For example, there can be about 500 to 5,000,000 platelets/μL, 1,000 to 4,000,000 platelets/μL, 5,000 to 3,500,000 platelets/μL, 10,000 to 3,000,000 platelets/μL, 50,000 to 2,500,000 platelets/μL, 100,000 to 2,500,000 platelets/μL, 500,000 to 2,500,000 platelets/μL, 750,000 to 2,500,000 platelets/μL, 1,000,000 to 2,500,000 platelets/μL, 1,000,000 to 2,250,000 platelets/μL, or 1,000,000 to 2,000,000 platelets/μL.

In some embodiments, presence of the activator leads to formation of platelet gel (also known as a platelet clot) in the silk/platelet composition. The addition of the activators induces the cleaving of fibrinogen to form fibrin which polymerizes, producing a glue-like gel. Platelets trapped in the gel are activated and release bioactive molecules. In some embodiments, a platelet gel can be formed and added into a silk fibroin solution for forming the silk/platelet composition disclosed herein. In some other embodiments, non-activated platelets can be co-added to the silk fibroin solution with a platelet activator. In some embodiments, activated platelets can be added to the silk fibroin solution before platelet gel has formed.

Platelets for use in the compositions disclosed herein can be obtained from any source available to the practitioner. For example, the platelets can be obtained from a single source (e.g. a single person) or combined from a plurality of sources (e.g., two or more people or group of people). For administration to a subject, the composition can comprise platelets that are autologous to the subject. For example, the platelet can be obtained from the subject and used for making the silk/platelet composition which the can be administered to the subject. In some situations, such as during emergency applications, platelets from another person or a group of people can be combined for forming the silk/platelet composition, which then can be administered to the subject in need thereof. For allogenic uses of the compositions disclosed herein, ABO compatibility between the subject and source of platelets is not mandatory, however, Rh matching can be useful. Without wishing to be bound by a theory, one advantage of silk/platelet composition disclosed herein can be that encapsulating the cells, e.g., platelets, in the silk/PG can avoid contact between recipient tissues and the cells encapsulated in the silk/PG.

In some embodiments, the platelets can be added to the silk fibroin as a platelet concentrate, e.g., platelet rich plasma. As used herein, the term “platelet concentrate” is a broad term which is used in its ordinary sense and is a concentration of platelets greater than the peripheral blood concentration suspended in a solution of plasma, or other excipient suitable for administration to a human or non-human animal including, but not limited to isotonic sodium chloride solution, physiological saline, normal saline, dextrose 5% in water, dextrose 10% in water, Ringer solution, lactated Ringer solution, Ringer lactate, Ringer lactate solution, and the like. The platelet concentrate can be an autologous preparation from whole blood taken from the subject to be treated or, alternatively, the platelet concentrate can be prepared from a whole blood sample taken from a single donor source or from whole blood samples taken from multiple donor sources. In all embodiments of this invention, the platelet concentrate is not Choukroun platelet-rich fibrin. The number of platelets can be counted manually or using an automatic counter. The automatic counter can also give information about the numbers of other blood cell components.

Depending on the leucocyte and fibrin content of the concentrate, platelet concentrates can be classified into four categories: pure PRP, leucocyte-rich PRP, pure platelet-rich fibrin (PRF), and leucocyte-rich PRF. For methods of production, see Ehrenfest et al., Cell 2009, 27, 158-167. The difference between PRP and PRF is that a PRP composition uses chemical additives including anticoagulants (e.g., heparin and citrate), coagulants (e.g., thrombin) and/or calcium salts (e.g., calcium chloride and calcium gluconate) during the production process, while a PRF composition does not use any chemical additive.

In some embodiments, the platelet concentrate is a pure PRP composition that is substantially free of leucocytes. The leucocytes can be separated from the platelets by centrifugation. As used herein, the term “substantially free” means that the ratio of leucocyte population over platelet population is less than 5%.

In some embodiments, the platelet concentrate is a leucocyte-rich PRP composition that further comprises leucocytes at a leucocyte concentration that is higher than the baseline concentration of the leucocytes in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration.

In some embodiments, the platelet concentrate is a pure PRF composition that is substantially free of leucocytes. In other embodiments, the platelet concentrate is a leucocyte-rich PRF composition that further comprises leucocytes at a leucocyte concentration that is higher than the baseline concentration of the leucocytes in whole blood.

In some embodiments, PRP compositions comprise lower levels of RBCs and hemoglobin relative to their baseline concentrations. In some embodiments of PRP composition, only the concentration of platelets is elevated relative to the baseline concentration. In some embodiments, PRP compositions comprise elevated concentrations of platelets and lower levels of neutrophils relative to their baseline concentrations. Some embodiments of PRP composition comprise elevated levels of platelets and neutrophil-depleted leucocytes compared to their baseline concentrations. In some embodiments of PRP, the ratio of lymphocytes and monocytes to neutrophils is significantly higher than the ratios of their baseline concentrations.

The PRP composition can include platelets at a level of between about 1.01 and about 2 times the baseline, about 2 and about 3 times the baseline, about 3 and about 4 times the baseline, about 4 and about 5 times the baseline; about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration can be between about 4 and about 6 times the baseline. Typically, a microliter of whole blood comprises at least 140,000 to 150,000 platelets and up to 400,000 to 500,000 platelets. The PRP compositions can comprise about 500,000 to about 7,000,000 platelets per microliter. In some instances, the PRP compositions can comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter.

In certain variations, the PRP composition can contain a specific concentration of neutrophils. The neutrophil concentration can vary between less than the baseline concentration of neutrophils to eight times than the baseline concentration of neutrophils. In some embodiments, the PRP composition can include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels can be depleted to 1% or less than that found in whole blood. In some variations, the neutrophil concentration can be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration can additionally or alternatively be specified relative to the concentration of the lymphocytes, monocytes, and/or eosinophil. One microliter of whole blood typically comprises 2,000 to 7,500 neutrophils. In some variations, the PRP composition can comprise neutrophils at a concentration of less than about 1,000 per microliter, about 1,000 to about 5,000 per microliter, about 5,000 to about 20,000 per microliter, about 20,000 to about 40,000 per microliter, or about 40,000 to about 60,000 per microliter. In some embodiments, neutrophils are eliminated or substantially eliminated. Means to deplete blood products, such as PRP, of neutrophils is known and discussed in U.S. Pat. No. 7,462,268, content of which is incorporated herein by reference in its entirety.

In certain variations, the PRP composition can contain a specific concentration of lymphocytes. The lymphocyte concentration can vary between less than the baseline concentration of lymphocytes to eight times than the baseline concentration of lymphocytes. In some embodiments, the PRP composition can include lymphocytes at a concentration of 50-70%, 30-50%, 10-30%, 5-10%, 1-5%, 0.5-1%, 0.1-0.5% of levels of lymphocytes found in whole blood or even less. In some embodiments, lymphocyte levels can be depleted to 1% or less than that found in whole blood. In some variations, the lymphocyte concentration can be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The lymphocyte concentration can additionally or alternatively be specified relative to the concentration of the neutrophils, monocytes and/or eosinophils. One microliter of whole blood typically comprises 1,300 to 4,000 lymphocytes. In some variations, the PRP composition can comprise lymphocytes at a concentration of less than about 1,000 per microliter, about 1,000 to about 5,000 per microliter, about 5,000 to about 20,000 per microliter, or about 20,000 to about 40,000 per microliter. In some embodiments, lymphocytes are eliminated or substantially eliminated.

In certain variations, the PRP composition can contain a specific concentration of monocytes. The monocyte concentration can vary between less than the baseline concentration of monocytes to eight times than the baseline concentration of monocytes. In some embodiments, the PRP composition can include monocytes at a concentration of 50-70%, 30-50%, 10-30%, 5-10%, 1-5%, 0.5-1%, 0.1-0.5% of levels of monocytes found in whole blood or even less. In some embodiments, monocyte levels can be depleted to 1% or less than that found in whole blood. In some variations, the monocyte concentration can be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The monocyte concentration can additionally or alternatively be specified relative to the concentration of the neutrophils, lymphocytes and/or eosinophils. One microliter of whole blood typically comprises 200 to 800 monocytes. In some variations, the PRP composition can comprise monocytes at a concentration of less than about 100 per microliter, about 100 to about 800 per microliter, about 800 to about 4,000 per microliter, or about 4,000 to about 8,000 per microliter. In some embodiments, monocytes are eliminated or substantially eliminated.

In certain variations, the PRP composition can contain a specific concentration of eosinophils. The eosinophil concentration can vary between less than the baseline concentration of eosinophils to eight times than the baseline concentration of eosinophils. In some embodiments, the PRP composition can include eosinophils at a concentration of 50-70%, 30-50%, 10-30%, 5-10%, 1-5%, 0.5-1%, 0.1-0.5% of levels of eosinophils found in whole blood or even less. In some embodiments, eosinophil levels can be depleted to 1% or less than that found in whole blood. In some variations, the eosinophil concentration can be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The eosinophil concentration can additionally or alternatively be specified relative to the concentration of the neutrophils, lymphocytes and/or monocytes. One microliter of whole blood typically comprises 40 to 400 eosinophils. In some variations, the PRP composition can comprise eosinophils at a concentration less than about 40 per microliter, about 30 to about 400 per microliter, about 400 to about 2,000 per microliter, or about 2,000 to about 5,000 per microliter. In some embodiments, eosinophils are eliminated or substantially eliminated.

In some embodiments, the PRP compositions can comprise a lower concentration of red blood cells (RBCs) and/or hemoglobin than the concentration in whole blood. The RBC concentration can be between about 0.01 and about 0.1 times baseline, about 0.1 and about 0.25 times baseline, about 0.25 and about 0.5 times baseline, or about 0.5 and about 0.9 times baseline. The hemoglobin concentration can be depressed and in some variations can be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women.

In some embodiments, the PRP compositions can comprise a lower concentration of leucocytes than the concentration in whole blood. The leucocyte concentration can be between about 0.01 and about 0.1 times baseline, about 0.1 and about 0.25 times baseline, about 0.25 and about 0.5 times baseline, or about 0.5 and about 0.9 times baseline. Typically, whole blood drawn from a male patient may have a leucocyte count of at least 4,100 to 4,500 and up to 10,900 to 11,000 per microliter.

In some embodiments, PRP composition can contain increased concentrations of growth factors and other cytokines. In some embodiments, PRP composition can include increased concentrations of one or more of: platelet-derived growth factor, transforming growth factor beta (β1, β2, β3), basic and acidic fibroblast growth factor (FGF2), insulin-like growth factor, insulin-like growth factor 2, vascular endothelial growth factor, epidermal growth factor, interleukin-8, keratinocyte growth factor, connective tissue growth factor, angiopoietin, adrenomedullin (AM), autocrine motility factor, bone morphogenetic proteins (BMPs), brain-derived neurotrophic factor (BDNF), erythropoietin (EPO), glial cell line-derived neurotrophic factor (GDNF), granulocyte colony-stimulating factor (G-CSF), granulocyte macrophage colony-stimulating factor (GM-CSF), growth differentiation factor-9 (GDF9), h growth factor (HGF), hepatoma-derived growth factor (HDGF), migration-stimulating factor, myostatin (GDF-8), nerve growth factor (NGF) and other neurotrophins, thrombopoietin (TPO), tumor necrosis factor-alpha (TNF-α), placental growth factor (PlGF), intrelukin-1, intrelukin-2, intrelukin-3, intrelukin-4, intrelukin-5, intrelukin-6, and intrelukin-7.

PRP can be added to a silk fibroin solution for forming the silk/platelet composition disclosed herein. Any ratio of PRP to the silk fibroin solution can be used. For example, the ratio of PRP to silk fibroin solution can range from about 50:1 to about 1:50. In some embodiments, the ratio can be from about 25:1 to about 1:25, from about 20:1 to about 1:20, from about 15:1 to about 1:15, from about 10:1 to about 1:10, from about 7.5:1 to about 1:75, from about 5:1 to about 1:5, from about 4:1 to about 1:4, from about 3:1 to about 1:3, from about 2.5:1 to about 1:2.5, from about 2:1 to about 1:2, or from about 1.5:1 to about 1:1.5. In some embodiments, the ratio is from about 1:1. In some embodiments, the ratio is based on volume.

In some embodiments, the platelets can be activated before formation of the silk/platelet composition described herein. For example, platelets in the PRP composition can be activated before the PRP composition is added to the silk fibroin solution for forming the silk/platelet composition. For example, the platelets in the PRP composition can be activated by thrombin and/or calcium before forming the silk/platelet composition described herein.

As used herein, the term “silk fibroin” or “fibroin” includes silkworm silk and insect or spider silk protein. See e.g., Lucas et al., Adv. Protein Chem. 1958, 13, 107-242. Any type of silk fibroin can be used according to aspects of the present invention. There are many different types of silk produced by a wide variety of species, including, without limitation: Antheraea mylitta; Antheraea pernyi; Antheraea yamamai; Galleria mellonella; Bombyx mori; Bombyx mandarins; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Other silks include transgenic silks, genetically engineered silks (recombinant silk), such as silks from bacteria, yeast, mammalian cells, transgenic animals, or transgenic plants, and variants thereof. See for example, WO 97/08315 and U.S. Pat. No. 5,245,012, content of both of which is incorporated herein by reference in its entirety. In some embodiments, silk fibroin can be derived from other sources such as spiders, other silkworms, bees, synthesized silk-like peptides, and bioengineered variants thereof. In some embodiments, silk fibroin can be extracted from a gland of silkworm or transgenic silkworms. See for example, WO2007/098951, content of which is incorporated herein by reference in its entirety.

In some embodiments, the composition comprises low molecular weight silk fibroin fragments, i.e., the composition comprises a population of silk fibroin fragments having a range of molecular weights, characterized in that: no more than 15% of total weight of the silk fibroin fragments in the population has a molecular weight exceeding 200 kDa, and at least 50% of the total weight of the silk fibroin fragments in the population has a molecular weight within a specified range, wherein the specified range is between about 3.5 kDa and about 120 kDa. Without limitations, the molecular weight can be the peak average molecular weight (Mp), the number average molecular weight (Mn), or the weight average molecular weight (Mw)

As used herein, the-phrase “silk fibroin fragments” refers to polypeptides having an amino acid sequence corresponding to fragments derived from silk fibroin protein, or variants thereof. In the context of the present disclosure, silk fibroin fragments generally refer to silk fibroin polypeptides that are smaller than the naturally occurring full length silk fibroin counterpart, such that one or more of the silk fibroin fragments within a population or composition are less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, etc. In some embodiments, “a composition comprising silk fibroin fragments” encompasses a composition comprising non-fragmented (i.e., full-length) silk fibroin polypeptide, in additional to shorter fragments of silk fibroin polypeptides. Silk fibroin fragments described herein can be produced as recombinant proteins, or derived or isolated (e.g., purified) from a native silk fibroin protein or silk cocoons. In some embodiments, the silk fibroin fragments can be derived by degumming silk cocoons under a specified condition selected to produce the silk fibroin fragments having the desired range of molecular weights. Low molecular weight silk fibroin compositions are described in US Provisional application titled “LOW MOLECULAR WEIGHT SILK FIBROIN AND USES THEREOF” filed on Sep. 27, 2013, and having attorney docket no. 700355-078010, content of which is incorporated herein by reference in its entirety.

In some embodiments, the silk fibroin is substantially depleted of its native sericin content (e.g., 5% (w/w) or less residual sericin in the final extracted silk). Alternatively, higher concentrations of residual sericin can be left on the silk following extraction or the extraction step can be omitted. In some embodiments, the sericin-depleted silk fibroin has, e.g., about 1% (w/w) residual sericin, about 2% (w/w) residual sericin, about 3% (w/w) residual sericin, about 4% (w/w), or about 5% (w/w) residual sericin. In some embodiments, the sericin-depleted silk fibroin has, e.g., at most 1% (w/w) residual sericin, at most 2% (w/w) residual sericin, at most 3% (w/w) residual sericin, at most 4% (w/w), or at most 5% (w/w) residual sericin. In some other embodiments, the sericin-depleted silk fibroin has, e.g., about 1% (w/w) to about 2% (w/w) residual sericin, about 1% (w/w) to about 3% (w/w) residual sericin, about 1% (w/w) to about 4% (w/w), or about 1% (w/w) to about 5% (w/w) residual sericin. In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected.

Without wishing to be bound by a theory, properties of the silk/platelet composition can be modify through controlled partial removal of silk sericin or deliberate enrichment of source silk with sericin. This can be accomplished by varying the conditions, such as time, temperature, concentration, and the like for the silk degumming process.

Degummed silk can be prepared by any conventional method known to one skilled in the art. For example, B. mori cocoons are boiled for about up to 90 minutes, generally about 10 to 60 minutes, in an aqueous solution. In one embodiment, the aqueous solution is about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution.

In some embodiments, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 5, 718-26, content of all which is incorporated herein by reference in their entirety. An exemplary organic solvent that can be used to produce a silk solution includes, but is not limited to, hexafluoroisopropanol (HFIP). See, for example, International Application No. WO2004/000915, content of which is incorporated herein by reference in its entirety. In some embodiments, the silk solution is entirely free or essentially free of organic solvents, i.e., solvents other than water.

The composition disclosed herein can comprise any amount/ratio of silk fibroin and any amount/ratio of platelets. Without wishing to be bound by a theory, the amount of silk fibroin in the solution used for making the silk/composition or the silk/platelet composition itself can be varied to vary properties of the silk/platelet composition. Generally, any amount of silk fibroin can be present in the solution used for making the silk/platelet composition. For example, amount of silk fibroin in the solution can be from about 0.1% (w/v) to about 90% (w/v). In some embodiments, the amount of silk fibroin in the solution can be from about 1% (w/v) to about 75% (w/v), from about 1% (w/v) to about 70% (w/v), from about 1% (w/v) to about 65% (w/v), from about 1% (w/v) to about 60% (w/v), from about 1% (w/v) to about 55% (w/v), from about 1% (w/v) to about 50% (w/v), from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the silk fibroin in the solution is about 25% (w/v). In some embodiments, the silk fibroin in the solution is about 0.5 (w/v) to about 30% (w/v), about 4% (w/v) to about 16% (w/v), about 4% (w/v) to about 14% (w/v), about 4% (w/v) to about 12% (w/v), about 4% (w/v) to about 0% (w/v), about 6% (w/v) to about 8% (w/v). Exact amount of silk in the silk solution can be determined by drying a known amount of the silk solution and measuring the mass of the residue to calculate the solution concentration.

Amount of silk fibroin in silk/platelet composition can be from about 1% (w/v) to about 90% (w/v). In some embodiments, the amount of silk fibroin in the silk/platelet composition can be from about 0.1% (w/v) to about 75% (w/v), from about 1% (w/v) to about 70% (w/v), from about 1% (w/v) to about 65% (w/v), from about 1% (w/v) to about 60% (w/v), from about 1% (w/v) to about 55% (w/v), from about 1% (w/v) to about 50% (w/v), from about 1% (w/v) to about 45% (w/v), from about 1% (w/v) to about 40% (w/v), from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the silk fibroin in the silk/platelet composition is about 25% (w/v). In some embodiments, the silk in the silk/platelet composition is about 0.5 (w/v) to about 30% (w/v), about 2% (w/v) to about 8% (w/v), about 2% (w/v) to about 7% (w/v), about 2% (w/v) to about 6% (w/v), about 2% (w/v) to about 5% (w/v), about 3% (w/v) to about 4% (w/v).

A confirmation change can be induced in the silk fibroin in the silk/platelet composition to make the silk fibroin at least partially insoluble. Without wishing to be bound by a theory, the induced conformational change alters the crystallinity of the silk fibroin, e.g., Silk II beta-sheet crystallinity. The conformational change can be induced by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al., Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-(3-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, the conformation of the silk fibroin can be altered by water annealing. Without wishing to be bound by a theory, it is believed that physical temperature-controlled water vapor annealing (TCWVA) provides a simple and effective method to obtain refined control of the molecular structure of silk biomaterials. The silk materials can be prepared with control of crystallinity, from a low content using conditions at 4° C. (α helix dominated silk I structure), to highest content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Temperature controlled water vapor annealing is described, for example, in Hu et al., Biomacromolecules, 2011, 12, 1686-1696, content of which is incorporated herein by reference in its entirety.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, the alteration in the conformation of the silk fibroin can be induced with sheer stress. The sheer stress can be applied, for example, by passing the silk composition through a needle. Other methods of inducing conformational changes include applying an electric field, applying pressure, or changing the salt concentration.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk is present completely in a silk II beta-sheet conformation, i.e., 100% silk II beta-sheet crystallinity.

In some embodiments, the silk fibroin in the silk/platelet composition has a protein structure that substantially includes β-turn and β-strand regions. Without wishing to be bound by a theory, the silk 0 sheet content can impact gel function and in invo longevity of the silk//platelet composition. It is to be understood that composition including non-β sheet content (e.g., e-gels) can also be utilized. In aspects of these embodiments, the silk fibroin in the silk/platelet composition has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the silk/platelet composition has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, the silk fibroin in the silk/platelet composition has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk 0 sheet content, from less than 10% to ˜55% can be used in the platelet gels.

In some embodiments, the silk fibroin in the silk/platelet composition has a protein structure that is substantially-free of α-helix and random coil regions. In aspects of these embodiments, the silk fibroin in the silk/platelet composition has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the silk/platelet composition has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the silk/platelet composition has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk fibroin in the silk/platelet composition has a protein structure that substantially includes β-turn and β-strand regions. In aspects of these embodiments, the silk fibroin in the silk/platelet composition has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the silk/platelet composition has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, the silk fibroin in the silk/platelet composition has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions.

In some embodiments, the silk fibroin in the silk/platelet composition has a protein structure that is substantially-free of α-helix and random coil regions. In aspects of these embodiments, the silk fibroin in the silk/platelet composition has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the silk/platelet composition has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the silk/platelet composition has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk/platelet composition disclosed herein is in the form of a fiber. As used herein, the term “fiber” means a relatively flexible, unit of matter having a high ratio of length to width across its cross-sectional perpendicular to its length. Methods for preparing silk fibroin fibers are well known in the art. A fiber can be prepared by electrospinning a silk solution, drawing a silk solution, and the like. Electrospun silk materials, such as fibers, and methods for preparing the same are described, for example in WO2011/008842, content of which is incorporated herein by reference in its entirety. Micron-sized silk fibers (e.g., 10-600 μm in size) and methods for preparing the same are described, for example in Mandal et al., PNAS, 2012, doi: 10.1073/pnas.1119474109; U.S. Provisional Application No. 61/621,209, filed Apr. 6, 2012; and PCT application no. PCT/US13/35389, filed Apr. 5, 2013, content of all of which is incorporated herein by reference.

It is noted that the fiber composition disclosed herein is different from and is novel and nonobvious over the electrospun scaffolds described in Sell et al. (Tissue Engineering, 2011, 17(21 and 22):2723-2737). First, Sell et al. uses lysed platelets in the silk solution used for electrospinning. In contrast, the composition disclosed herein comprises unlysed platelets. Second, Sell et al. shows a general decrease in scaffold mechanical properties when loaded with the powdered PRP (lysed platelets). In contrast, the composition disclosed herein can be created with different mechanical properties by controlling, for example, silk concentration, sterilization, and silk boiling time. This allows the compositions disclosed herein to be used for different applications by modulating the mechanical properties. Third, Sell et al. uses hexafluoroisopropanol (HFIP) as the electorspinning solvent. In contrast, the silk/platelet composition disclosed herein can be prepared using water. This allows one to form compositions for in vitro, ex-vivo, or in vivo use without having to worry about any toxic or adverse effects relating to use of organic solvents.

In some embodiments, the silk/platelet composition disclosed herein can be in the form of a film, e.g., a silk film. As used herein, the term “film” refers to a flat or tubular flexible structure. It is to be noted that the term “film” is used in a generic sense to include a web, film, sheet, laminate, or the like. In some embodiments, the film is a patterned film, e.g., nanopatterned film. Exemplary methods for preparing silk fibroin films are described in, for example, WO 2004/000915 and WO 2005/012606, content of both of which is incorporated herein by reference in its entirety.

It is noted that the film composition disclosed herein is different from and is novel and nonobvious over the silk film as described in Motta et al. (J. Biomater. Sci. Polym. Ed, 2009, 20(13): 1857-97. Motta et al. describes adsorption of platelet gel onto the film surface and not encapsulation into the films. In contrast, in the compositions disclosed herein, platelet gel is encapsulated in the silk film. This allows for storage of the silk platelet for controlled release/function.

In some embodiments, the silk/platelet composition disclosed herein can be in the form of a cylindrical matrix, e.g., a silk tube. The silk tubes can be made using any method known in the art. For example, tubes can be made using molding, dipping, electrospinning, gel spinning, and the like. Gel spinning is described in Lovett et al. (Biomaterials 2008, 29(35):4650-4657) and the construction of gel-spun silk tubes is described in PCT application no. PCT/US2009/039870, filed Apr. 8, 2009, content of both of which is incorporated herein by reference in their entirety. Construction of silk tubes using the dip-coating method is described in PCT application no. PCT/US2008/072742, filed Aug. 11, 2008, content of which is incorporated herein by reference in its entirety. Construction of silk fibroin tubes using the film-spinning method is described in PCT application No. PCT/US2013/030206, filed Mar. 11, 2013 and U.S. Provisional application No. 61/613,185, filed Mar. 20, 2012.

It is noted that the tube composition disclosed herein is different from and is novel and nonobvious over the silk tube as described in Lovett et al. (Organogenesis 2010, 6(4): 217-224). Lovett et al. describes the use of silk tubes without PRP for small diameter vascular grafts. While Lovett el al. incubates PRP on silk to demonstrate that silk does not activate platelets, they do not teach any composition that includes both silk and PRP. In contrast, the compositions disclosed herein include both silk and PRP, in which the PRP can release growth factors for wound healing.

In some embodiments, the silk/platelet composition disclosed herein can be a porous matrix or scaffold. For example, the porous scaffold can have a porosity of at least about 10%, at least about 20%, at least about 30%, at least about 40%, at least about 50%, at least about 60%, at least about 70%, at least about 80%, at least about 90%, or higher. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

The porous scaffold can have any pore size. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section.

Methods for forming pores in silk fibroin-based scaffolds are known in the art and include, but are not limited, porogen-leaching methods, freeze-drying methods, and/or gas-forming method. Exemplary methods for forming pores in a silk-based material are described, for example, in U.S. Pat. App. Pub. Nos.: US 2010/0279112 and US 2010/0279112; U.S. Pat. No. 7,842,780; and WO2004062697, content of all of which is incorporated herein by reference in its entirety.

Though not meant to be bound by a theory, a porous scaffold's porosity, structure, and mechanical properties can be controlled via different post-spinning processes such as vapor annealing, heat treatment, alcohol treatment, air-drying, lyophilization and the like. Additionally, any desirable release rates, profiles or kinetics of a molecule encapsulated in the matrix can be controlled by varying processing parameters, such as matrix thickness, silk molecular weight, concentration of silk in the matrix, beta-sheet conformation structures, silk II beta-sheet crystallinity, or porosity and pore sizes.

In some embodiments, silk/PG can be at least partially dried (e.g., lyophilization). This can provide long-term storage of the silk/PG materials. For example, if one needs silk/PG on demand, one can keep a powder format on-demand and initiate gelation at the site of implantation.

In some embodiments, the silk composition can further comprise one or more (e.g., one, two, three, four, five or more) additives. Without wishing to be bound by a theory additive can provide one or more desirable properties, e.g., strength, flexibility, ease of processing and handling, biocompatibility, bioresorability, surface morphology, release rates and/or kinetics of one or more factors released by the platelets, and the like. The additive can be covalently or non-covalently linked with silk fibroin and can be integrated homogenously or heterogeneously within the silk composition.

Without limitations, the additive can be selected from small organic or inorganic molecules; saccharines; oligosaccharides; polysaccharides; biological macromolecules, e.g., peptides, proteins, and peptide analogs and derivatives; peptidomimetics; antibodies (polyclonal and monoclonal) and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. Furthermore, the additive can be in any physical form. For example, the additive can be in the form of a particle, a fiber, a film, a gel, a mesh, a mat, a non-woven mat, a powder, a liquid, or any combinations thereof. In some embodiments, the additive is a particle.

Total amount of additives in the composition can be from about 0.01 wt % to about 99 wt %, from about 0.01 wt % to about 70 wt %, from about 5 wt % to about 60 wt %, from about 10 wt % to about 50 wt %, from about 15 wt % to about 45 wt %, or from about 20 wt % to about 40 wt %, of the total silk composition. In some embodiments, ratio of silk fibroin to additive in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, the silk/platelet composition comprises a molar ratio of silk fibroin to the additive of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, the silk/platelet composition comprises a molar ratio of silk fibroin to the additive of, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, the silk/platelet composition comprises a molar ratio of silk fibroin to the additive of e.g., from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

In some embodiments, the additive is a biologically active agent. The term “biologically active agent” as used herein refers to any molecule which exerts at least one biological effect in vivo. For example, the biologically active agent can be a therapeutic agent to treat or prevent a disease state or condition in a subject. Biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be incorporated into the composition described herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

Generally, any therapeutic agent can be included in the composition described herein. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, shRNA, aptamers, ribozymes, decoy nucleic acids, antisense nucleic acids, RNA activators, and the like.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain one therapeutic agent or combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the therapeutic agent is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N°-monomethyl-Larginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof

Prostaglandins are art recognized and are a class of naturally occurring chemically related, long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell. Trophic factors include, Without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

In some embodiments, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-β, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

In some embodiments, the silk/platelet composition can further comprise at least one additional material for soft tissue augmentation, e.g., dermal filler materials, including, but not limited to, poly(methyl methacrylate) microspheres, hydroxylapatite, poly(L-lactic acid), collagen, elastin, and glycosaminoglycans, hyaluronic acid, commerical dermal filler products such as BOTOX® (from Allergan), DYSPORT®, COSMODERM®, EVOLENCE®, RADIESSE®, RESTYLANE®, JUVEDERM® (from Allergan), SCULPTRA®, PERLANE®, and CAPTIQUE®, and any combinations thereof.

In some embodiments, the additive is a wound healing agent. As used herein, a “wound healing agent” is a compound or composition that actively promotes wound healing process. Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as sphingosine-1-phosphate and lysophosphatidic acid; amino acids, such as arginine and lysine; peptides such as the bradykinins, substance P and calcium gene-related peptide (CGRP); nitric oxide; and any combinations thereof.

In some embodiments, the additive is a cell, e.g., a biological cell. It is to be understood that the cell in the form of the additive is in addition to the platelets present in the composition. Cells useful for incorporation into the composition can come from any source, e.g., mammalian, insect, plant, etc. In some embodiments, the cell can be a human, rat or mouse cell. In general, cells to be used with the compositions described herein can be any types of cells. In general, the cells should be viable when encapsulated within compositions. In some embodiments, cells that can be used with the composition include, but are not limited to, mammalian cells (e.g. human cells, primate cells, mammalian cells, rodent cells, etc.), avian cells, fish cells, insect cells, plant cells, fungal cells, bacterial cells, and hybrid cells. In some embodiments, exemplary cells that can be can be used with the compositions include stem cells, totipotent cells, pluripotent cells, and/or embryonic stem cells. In some embodiments, exemplary cells that can be encapsulated within compositions include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some embodiments, the cell can be a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used. For example, human skin cells reprogrammed into embryonic stem cells by the transduction of Oct3/4, Sox2, c-Myc and Klf4 (Junying Yu, et. al., Science, 2007, 318, 1917-1920 and Takahashi K. et. al., Cell, 2007, 131, 1-12).

Inventors have discovered that release of platelet-derived factors from the silk/platelet composition can be controlled adding agents to the composition that can disrupt interactions between silk fibroin and the factor. Accordingly, in some embodiments, the additive an agent that disrupts, inhibits, or reduces protein-factor interactions. For example, silk fibroin has a net negative charge. Thus, factors having a net positive charge can interact with silk fibroin through ionic/electrostatic interactions and be retained in the silk/platelet composition. Altering the ionic/electrostatic interactions can change the release rate of the factor from the composition. One way of altering ionic/electrostatic interactions can be by adding a cationic molecule to the silk/platelet composition. Some other factors can interact with silk fibroin through hydrophobic interactions. Without wishing to be bound by a theory, such factors can be released using agents that disrupt, inhibit, or reduce non-ionic interactions. Thus, in some embodiments, the additive can be a surfactant. As used herein, the term “surfactant” refers to a natural or synthetic amphiphilic compound. A surfactant can be non-ionic, zwitterionic, or ionic. Non-limiting examples of surfactants include polysorbates like polysorbate 20 (TWEEN® 20), polysorbate 40 (TWEEN® 40), polysorbate 60 (TWEEN® 60), polysorbate 61 (TWEEN® 61), polysorbate 65 (TWEEN® 65), polysorbate 80 (TWEEN® 80), and polysorbate 81 (TWEEN® 81); poloxamers (polyethylene-polypropylene copolymers), like Poloxamer 124 (PLURONIC® L44), Poloxamer 181 (PLURONIC® L61), Poloxamer 182 (PLURONIC® L62), Poloxamer 184 (PLURONIC® L64), Poloxamer 188 (PLURONIC® F68), Poloxamer 237 (PLURONIC® F87), Poloxamer 338 (PLURONIC® L108), Poloxamer 407 (PLURONIC® F127), polyoxyethyleneglycol dodecyl ethers, like BRIJ® 30, and BRIJ® 35; 2-dodecoxyethanol (LUBROL®-PX); polyoxyethylene octyl phenyl ether (TRITON® X-100); sodium dodecyl sulfate (SDS); 3-[(3-Cholamidopropyl)dimethylammonio]-1-propanesulfonate (CHAPS); 3-[(3-Cholamidopropyl)dimethylammonio]-2-hydroxy-1-propanesulfonate (CHAPSO); sucrose monolaurate; and sodium cholate. Other non-limiting examples of surfactant excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety. In some embodiments, the surfactant is a cationic polymer. In one embodiment, the cationic polymer is polylysine, e.g., ε-poly-L-lysine. In alternative embodiments, the surfactant is an anionic polymer. In one embodiment, the anionic polymer is polyglutamate, e.g., poly-L-glutamate.

Another way of altering the ionic/electrostatic interactions can be by modulating the net negative charge of the silk fibroin. This can be accomplished by derivatizing or modifying silk with positively/negatively charged molecules. In some embodiments, the silk fibroin can be modified with positively/negatively charged peptides or polypeptides, such poly-lysine and poly-glutamic acid. While possible, it is not required that every single silk fibroin molecule in the composition be modified with a positively/negatively charged molecule. Methods of derivatizing or modifying silk fibroin with charged molecules are described in, for example, PCT application no. PCT/US2011/027153, filed Mar. 4, 2011, content of which is incorporated herein by reference in its entirety.

Ratio of modified silk fibroin to unmodified silk fibroin can be adjusted to optimize one or more desired properties of the composition, such as growth factor release. Accordingly, in some embodiments, ratio of modified to unmodified silk fibroin in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, the silk/platelet composition comprises a molar ratio of modified to unmodified silk fibroin of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, the silk/platelet composition comprises a molar ratio of modified to unmodified silk fibroin of, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, the silk/platelet composition comprises a molar ratio of modified to unmodified silk fibroin of e.g., from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

Another way of altering the release of factors is to change the porosity of the silk matrix. Porosity of the matrix can impact diffusion rate and the release kinetics. Methods for forming pores in silk fibroin-based scaffolds are previously described. Two or more ways of altering the release of factors can be used in combination. For example, one can control the net charge of the silk fibroin and the matrix porosity in a silk fibroin-based scaffold.

In some embodiments, the composition further comprises silk particles. The silk particles can be nanoparticles or microparticles. As used herein, the term “particle” includes spheres; rods; shells; and prisms; and these particles can be part of a network or an aggregate. Without limitations, the particle can have any size from nm to millimeters. In some embodiments, the particles can have a size ranging from about 0.01 μm to about 1000 μm, about 0.05 μm to about 500 μm, about 0.1 μm to about 250 μm, about 0.25 μm to about 200 μm, or about 0.5 μm to about 100 μm. Further, the silk particle can be of any shape or form, e.g., spherical, rod, elliptical, cylindrical, capsule, or disc. In some embodiments, the silk particle is a microparticle or a nanoparticle. As used herein, the term “microparticle” refers to a particle having a particle size of about 1 μm to about 1000 μm. As used herein, the term “nanoparticle” refers to particle having a particle size of about 0.1 nm to about 1000 nm.

Without wishing to be bound by a theory, particle size can greatly determine microscopic and macroscopic properties of the final product. Particle size is dependent on a number of process parameters, including, but not limited to, the size of the ceramic balls used, the amount of silk placed in each ball mill cup, the rotational speed (RPM) of the machine, and the duration of ball milling. Particle size in the powder can be predicted based on some of these process parameters, e.g., with mathematical modeling and/or experimentation to determine the correlation. For example, this can be done by milling a given volume of silk fibroin for varying ball mill speeds and durations. Scanning Electron Microscopy (SEM) can be performed on representative samples from each experiment to determine particle size. Additional tests can be run on each sample to determine the effect of process parameters on the color, molecular weight, viscosity in a solution, and solubility in water of the resulting constructs.

Various methods of producing silk particles (e.g., nanoparticles and microparticles) are known in the art. In some embodiments, the silk particles can be produced by a polyvinyl alcohol (PVA) phase separation method as described in, e.g., International App. No. WO 2011/041395, the content of which is incorporated herein by reference in its entirety. Other methods for producing silk fibroin particles are described, for example, in U.S. App. Pub. No. U.S. 2010/0028451 and PCT App. Pub. No.: WO 2008/118133 (using lipid as a template for making silk microspheres or nanospheres), and in Wenk et al. J Control Release, Silk fibroin spheres as a platform for controlled drug delivery, 2008; 132: 26-34 (using spraying method to produce silk microspheres or nanospheres), content of all of which is incorporated herein by reference in its entirety.

In some embodiments, silk particles can be produced using a freeze-drying method as described in U.S. Provisional Application Ser. No. 61/719,146, filed Oct. 26, 2012, content of which is incorporated herein by reference in its entirety. Specifically, silk foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure. After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

In some embodiments, the additive can be a silk-based material. The silk-based material can be selected from the group consisting of silk fibers, micro-sized silk fibers, unprocessed silk fibers, silk particles, and any combinations thereof. In some embodiments, the additive is a silk fiber. While the use of silk fibers is described in for example, US patent application publication no. US20110046686, the previously described materials do not provide machinable silk materials as disclosed in the present disclosure.

In some embodiments, the silk fibers are microfibers or nanofibers. In some embodiments, the additive is micron-sized silk fiber (10-600 μm). Micron-sized silk fibers can be obtained by hydrolyzing the degummed silk fibroin or by increasing the boing time of the degumming process. Alkali hydrolysis of silk fibroin to obtain micron-sized silk fibers is described for example in Mandal et al., PNAS, 2012, doi: 10.1073/pnas.1119474109; U.S. Provisional Application No. 61/621,209, filed Apr. 6, 2012; and PCT application no. PCT/US13/35389, filed Apr. 5, 2013, content of all of which is incorporated herein by reference. Because regenerated silk fibers made from HFIP silk solutions are mechanically strong, the regenerated silk fibers can also be used as additive.

In some embodiments, the silk fiber is an unprocessed silk fiber, e.g., raw silk or raw silk fiber. The term “raw silk” or “raw silk fiber” refers to silk fiber that has not been treated to remove sericin, and thus encompasses, for example, silk fibers taken directly from a cocoon Thus, by unprocessed silk fiber is meant silk fibroin, obtained directly from the silk gland. When silk fibroin, obtained directly from the silk gland, is allowed to dry, the structure is referred to as silk I in the solid state. Thus, an unprocessed silk fiber comprises silk fibroin mostly in the silk I conformation. A regenerated or processed silk fiber on the other hand comprises silk fibroin having a substantial silk II or beta-sheet crystallinity.

In some embodiments, the additive is a biocompatible polymer. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), polyphosphate ester), polycaprolactone, gelatin, collagen, fibronectin, keratin, polyaspartic acid, alginate, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, alginate, polyaspartic acid, any derivatives thereof and any combinations thereof. Other exemplary biocompatible polymers amenable to use according to the present disclosure include those described for example in U.S. Pat. No. 6,302,848; No. 6,395,734; No. 6,127,143; No. 5,263,992; No. 6,379,690; No. 5,015,476; No. 4,806,355; No. 6,372,244; No. 6,310,188; No. 5,093,489; No. 387,413; No. 6,325,810; No. 6,337,198; No. U.S. Pat. No. 6,267,776; No. 5,576,881; No. 6,245,537; No. 5,902,800; and No. 5,270,419, content of all of which is incorporated herein by reference. As used herein, the term “biocompatible” refers to a material that does not elicit a substantial immune response in the host.

In some embodiments, the biocompatible polymer is PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, the additive is an enzyme that hydrolyzes silk fibroin. Without wishing to be bound by a theory, such enzymes can be used to control the degradation of the article of manufacture.

In some embodiments, o recover or induce mass release of additives entrapped inside a silk-based construct (e.g., PG/Silk) wherein the silk fibroin has undergone conformational changes, LiBr or urea can be used to dissolve the silk fibroin matrix.

In some embodiments, the silk/platelet composition is bioresorbable. By “bioresorbable” is meant the ability of a material to be resorbed or remodeled in vivo. The resorption process involves degradation and elimination of the original implant material through the action of body fluids, enzymes or cells. The resorbed materials can be used by the host in the formation of new tissue, or it can be otherwise re-utilized by the host, or it can be excreted. The silk/platelet composition disclosed herein can have a resorption half-life of approximately 6 months to approximately 12 months. In some embodiments, the silk/platelet composition disclosed herein has a resorption half-life of approximately 9 months. The silk/platelet composition disclosed herein can be completely resorbed in approximately 12 months to approximately 24 months. In some embodiments the material is completely resorbed in approximately 12 months.

In some embodiments, the silk/platelet composition is in form of an injectable composition. As used herein, the term “injectable composition” generally refers to a composition that can be delivered or administered into a tissue with a minimally invasive procedure. The term “minimally invasive procedure” refers to a procedure that is carried out by entering a subject's body through the skin or through a body cavity or an anatomical opening, but with the smallest damage possible (e.g., a small incision, injection). In some embodiments, the injectable composition can be administered or delivered into a tissue by injection. In some embodiments, the injectable composition can be delivered into a tissue through a small incision on the skin followed by insertion of a needle, a cannula, and/or tubing, e.g., a catheter. Without wishing to be limited, the injectable composition can be administered or placed into a tissue by surgery, e.g., implantation.

In some embodiments, the injectable compositions can further comprise a pharmaceutically acceptable carrier. The compositions suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g. PBS).

Additionally, various additives which enhance the stability, sterility, and isotonicity of the injectable compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like. The injectable compositions can also contain auxiliary substances such as wetting or emulsifying agents, pH buffering agents, gelling or viscosity enhancing additives, preservatives, colors, and the like, depending upon the preparation desired.

Viscosity of the injectable compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected, and the desired viscosity for injection. The important point is to use an amount which will achieve the selected viscosity, e.g., addition of such thickening agents into some embodiments of the injectable compositions.

In some embodiments, the silk/platelet composition is in form of a sprayable composition. In some embodiments, the silk/platelet composition is in form of a powder.

The silk/platelet composition can have hardness, compressive strength, compressive toughness, resistance to deformation, compressive elastic modulus, and/or other mechanical properties optimized for the desired use. Accordingly, in some embodiments, the silk/platelet composition has hardness. Hardness refers to various properties of an object in the solid phase that gives it high resistance to various kinds of shape change when force is applied. Hardness is measured using a durometer and is a unitless value that ranges from zero to 100. The ability or inability of silk/platelet composition to be easily compressed can affect its suitability for application in different tissue replacement roles, i.e., mechanical compliance as bone, fat, connective tissue. Hardness can also affect the ability of the composition to be effectively comminuted, the reason being that a hard material can be more easily and consistently comminuted. Hardness can also affect extrudability, as a soft material can be more readily able to be slightly compressed during injection to pack with other particles or change shape to pass through a syringe barrel or needle.

In some embodiments, the silk/platelet composition exhibits low hardness. In aspects of these embodiments, the silk/platelet composition exhibits a hardness of, e.g., about 5, about 10, about 15, about 20, about 25, about 30, or about 35. In other aspects of these embodiments, the silk/platelet composition exhibits a hardness of, e.g., at most 5, at most 10, at most 15, at most 20, at most 25, at most 30, or at most 35. In yet other aspects of these embodiments, the silk/platelet composition exhibits a hardness of, e.g., about 5 to about 35, about 10 to about 35, about 15 to about 35, about 20 to about 35, or about 25 to about 35, about 5 to about 40, about 10 to about 40, about 15 to about 40, about 20 to about 40, about 25 to about 40, or about 30 to about 40.

In some embodiments, the silk/platelet composition exhibits medium hardness. In aspects of these embodiments, the silk/platelet composition exhibits a hardness of, e.g., about 40, about 45, about 50, about 55, or about 60. In other aspects of these embodiments, the silk/platelet composition exhibits a hardness of, e.g., at least 40, at least 45, at least 50, at least 55, or at least 60. In yet other aspects of these embodiments, the silk/platelet composition exhibits a hardness of, e.g., at most 40, at most 45, at most 50, at most 55, or at most 60. In still other aspects of these embodiments, the silk/platelet composition exhibits a hardness of, e.g., about 35 to about 60, about 35 to about 55, about 35 to about 50, about 35 to about 45, about 40 to about 60, about 45 to about 60, about 50 to about 60, about 55 to about 60, about 40 to about 65, about 45 to about 65, about 50 to about 65, about 55 to about 65.

In some embodiments, the silk/platelet composition exhibits high hardness. In aspects of these embodiments, the silk/platelet composition exhibits a hardness of, e.g., about 65, about 70, about 75, about 80, about 85, about 90, about 95, or about 100. In other aspects of these embodiments, the silk/platelet composition exhibits a hardness of, e.g., at least 65, at least 70, at least 75, at least 80, at least 85, at least 90, at least 95, or at least 100. In yet other aspects of these embodiments, the silk/platelet composition exhibits a hardness of, e.g., about 65 to about 100, about 70 to about 100, about 75 to about 100, about 80 to about 100, about 85 to about 100, about 90 to about 100, about 65 to about 75, about 65 to about 80, about 65 to about 85, about 65 to about 90, about 65 to about 95, about 60 to about 75, about 60 to about 80, about 60 to about 85, about 60 to about 90, or about 60 to about 95.

In some embodiments, the silk/platelet composition exhibits high resistance to deformation. In some embodiments, the silk/platelet composition exhibits low resistance to deformation. Deformable silk compositions, without platelets, are described for example, in WO2010/042798.

In some embodiments, the silk/platelet composition exhibits an elastic modulus. In some embodiments, the silk/platelet composition exhibits a high elastic modulus. In some other embodiments, the silk/platelet composition exhibits a low elastic modulus. Elastic modulus, or modulus of elasticity, refers to the ability of a material to resists deformation, or, conversely, an object's tendency to be non-permanently deformed when a force is applied to it. The elastic modulus of an object is defined as the slope of its stress-strain curve in the elastic deformation region: X=stress/strain, where X is the elastic modulus in Pascal's; stress is the force causing the deformation divided by the area to which the force is applied; and strain is the ratio of the change caused by the stress to the original state of the object. Specifying how stresses are to be measured, including directions, allows for many types of elastic moduli to be defined. The three primary elastic moduli are tensile modulus, shear modulus, and bulk modulus.

Tensile modulus (E) or Young's modulus is an objects response to linear strain, or the tendency of an object to deform along an axis when opposing forces are applied along that axis. It is defined as the ratio of tensile stress to tensile strain. It is often referred to simply as the elastic modulus. The shear modulus or modulus of rigidity refers to an object's tendency to shear (the deformation of shape at constant volume) when acted upon by opposing forces. It is defined as shear stress over shear strain. The shear modulus is part of the derivation of viscosity. The shear modulus is concerned with the deformation of a solid when it experiences a force parallel to one of its surfaces while its opposite face experiences an opposing force (such as friction). The bulk modulus (K) describes volumetric elasticity or an object's resistance to uniform compression, and is the tendency of an object to deform in all directions when uniformly loaded in all directions. It is defined as volumetric stress over volumetric strain, and is the inverse of compressibility. The bulk modulus is an extension of Young's modulus to three dimensions.

In some embodiments, the silk/platelet composition exhibits a tensile modulus. In aspects of these embodiments, the silk/platelet composition exhibits a tensile modulus of, e.g., about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa. In other aspects of these embodiments, the silk/platelet composition exhibits a tensile modulus of, e.g., at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa In yet other aspects of these embodiments, the silk/platelet composition exhibits a tensile modulus of, e.g., about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa.

In some embodiments, the silk/platelet composition exhibits shear modulus. In aspects of these embodiments, the silk/platelet composition exhibits a shear modulus of, e.g., about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa. In other aspects of these embodiments, the silk/platelet composition exhibits a shear modulus of, e.g., at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa In yet other aspects of these embodiments, the silk/platelet composition exhibits a shear modulus of, e.g., about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa.

In some embodiments, the silk/platelet composition exhibits a bulk modulus. In aspects of these embodiments, the silk/platelet composition exhibits exhibits a bulk modulus of, e.g., about 5 GPa, about 6 GPa, about 7 GPa, about 8 GPa, about 9 GPa, about 10 GPa, about 15 GPa, about 20 GPa, about 25 GPa, about 30 GPa, about 35 GPa, about 40 GPa, about 45 GPa, about 50 GPa, about 60 GPa, about 70 GPa, about 80 GPa, about 90 GPa, about 100 GPa. In other aspects of these embodiments, the silk/platelet composition exhibits exhibits a bulk modulus of, e.g., at least 5 GPa, at least 6 GPa, at least 7 GPa, at least 8 GPa, at least 9 GPa, at least 10 GPa, at least 15 GPa, at least 20 GPa, at least 25 GPa, at least 30 GPa, at least 35 GPa, at least 40 GPa, at least 45 GPa, at least 50 GPa, at least 60 GPa, at least 70 GPa, at least 80 GPa, at least 90 GPa, at least 100 GPa. In yet other aspects of these embodiments, the silk/platelet composition exhibits exhibits a bulk modulus of, e.g., about 5 GPa to about 50 GPa, about 5 GPa to about 100 GPa, about 10 GPa to about 50 GPa, about 10 GPa to about 100 GPa, or about 50 GPa to about 100 GPa.

In some embodiments, the silk/platelet composition exhibits high tensile strength. Tensile strength has three different definitional points of stress maxima. Yield strength refers to the stress at which material strain changes from elastic deformation to plastic deformation, causing it to deform permanently. Ultimate strength refers to the maximum stress a material can withstand when subjected to tension, compression or shearing. It is the maximum stress on the stress-strain curve. Breaking strength refers to the stress coordinate on the stress-strain curve at the point of rupture, or when the material pulls apart.

In some embodiments, the silk/platelet composition exhibits high yield strength relative to other polymer classes. In aspects of these embodiments, an elastomer matrix defining an array of interconnected pores exhibits a yield strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of these embodiments, the silk/platelet composition exhibits a yield strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of these embodiments, the silk/platelet composition exhibits a yield strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of these embodiments, the silk/platelet composition exhibits a yield strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

In some embodiments, the silk/platelet composition exhibits high ultimate strength. In aspects of these embodiments, the silk/platelet composition exhibits an ultimate strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of these embodiments, the silk/platelet composition exhibits an ultimate strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of these embodiments, the silk/platelet composition exhibits an ultimate strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of these embodiments, the silk/platelet composition exhibits an ultimate strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

In some embodiments, the silk/platelet composition exhibits high breaking strength. In some embodiments, the silk/platelet composition exhibits low breaking strength. In aspects of these embodiments, the silk/platelet composition exhibits a breaking strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of these embodiments, the silk/platelet composition exhibits a breaking strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of these embodiments, the silk/platelet composition exhibits a breaking strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of these embodiments, the silk/platelet composition exhibits a breaking strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

The physical and/or mechanical properties of silk/platelet compositions can be tuned by varying one or more of the following parameters: (i) amount and/or concentration of silk in the composition; (ii) amount and/or concentration of platelets (e.g., platelet gel) in the composition; (iii) ratio of amount of silk to platelets (e.g., platelet gel) in the composition; (iv) silk fibroin concentration of the solution used to make the composition; (v) molecular weight of the silk in the composition; (vi) degumming time, e.g., cocoon boiling time, of silk used in the composition; (vii) amount of any additives in the composition; (viii) net negative charge of silk in the composition; (ix) conformation of silk in the composition; (x) method of forming the silk/PG gel; and (xi) method of inducing platelet gel formation in the composition. Without wishing to be bound by a theory, it is believed that higher silk fibroin concentration can lead to stronger silk/platelet compositions. Shorter silk cocoon boiling time (e.g., high molecular weight silk) can also lead to stronger silk/platelet compositions. The mechanical properties can be varied depending on the applications.

In view of the above, the disclosure also provides a method of modulating a physical or mechanical property of a composition disclosed herein. Generally the method comprising varying a parameter selected from the group consisting of: (i) amount and/or concentration of silk in the composition; (ii) amount and/or concentration of platelets (e.g., platelet gel) in the composition; (iii) ratio of amount of silk to platelets (e.g., platelet gel) in the composition; (iv) silk fibroin concentration of the solution used to make the composition; (v) molecular weight of the silk in the composition; (vi) degumming time, e.g., cocoon boiling time, of silk used in the composition; (vii) amount of any additives in the composition; (viii) net negative charge of silk in the composition; (ix) conformation of silk in the composition; (x) method of forming the silk/PG gel; (xi) method of inducing platelet gel formation in the composition; and (xii) any combinations thereof. The physical or mechanical property can selected from stiffness, transparency, hardness, resistance to deformation, elastic modulus (e.g., tensile modulus, shear modulus, and bulk modulus), tensile strength, yield strength, ultimate strength, breaking strength, release rate or kinetics of agents such as growth factors, and any combinations thereof.

Without limitations, release of growth factors from a silk based composition can be modulated using the method disclosed herein for modulating a physical or mechanical property of the composition. Furthermore, the degradation properties of a composition disclosed herein, e.g., in form of an implant, can also be modulated using the method disclosed herein for modulating a physical or mechanical property of the composition.

For administration to a subject, the silk/platelet composition can be formulated in pharmaceutically acceptable compositions which comprise the silk/platelet composition disclosed herein, formulated together with one or more pharmaceutically acceptable carriers (additives) and/or diluents. The pharmaceutical composition can be specially formulated for administration in solid or liquid form, including those adapted for the following: (1) topical application, for example, as a cream, ointment, a controlled-release patch, or spray applied to the skin; (2) parenteral administration, for example, by subcutaneous, intramuscular, intravenous, or epidural injection as, for example, a sterile solution or suspension, or sustained-release formulation; (3) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (4) intravaginally or intrarectally, for example, as a pessary, cream or foam; (5) sublingually; (6) ocularly; (7) transdermally; (8) transmucosally; or (9) nasally. Additionally, compositions can be implanted into a patient or injected using a drug delivery composition. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

As used herein, the term “administered” refers to the placement of a drug delivery composition into a subject by a method or route which results in at least partial localization of the pharmaceutically active agent at a desired site.

The composition can be administered by any appropriate route which results in effective treatment in the subject, i.e. administration results in delivery to a desired location in the subject where at least a portion of the pharmaceutically active agent is delivered. Exemplary modes of administration include, but are not limited to, topical, implant, injection, infusion, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In some embodiments, the silk/platelet composition disclosed herein can be implanted in a subject. As used herein, the term “implanted,” and grammatically related terms, refers to the positioning of the silk/platelet composition in a particular locus in the subject, either temporarily, semi-permanently, or permanently. The term does not require a permanent fixation of the silk/platelet composition in a particular position or location. Exemplary in vivo loci include, but are not limited to site of a wound, trauma or disease.

### Exemplary Uses

The silk/platelet composition disclosed herein can be used in a variety of medical fields: wound healing, orthopedics, dentistry, rheumatology, dermatology, and the like. The compositions can be used in different fields of regenerative medicine and tissue repair. In particular, the silk/platelet composition disclosed herein can improve the treatment of chronic or difficult wound such as diabetic ulcers or extended tissue damages or bone loss in dentistry and orthopedics. Further, the spray or coating methods of application and delivery can improve the accessibility and the use of the composition to patients for skin repair.

Accordingly, in some embodiments, the silk/platelet composition disclosed herein can be used for repairing or augmenting a tissue in a subject. For example, the composition can be used for wound repair, soft tissue repair or augmentation, fillers for tissue space, templates for tissue reconstruction or regeneration, scaffolds for cells in tissue engineering applications, or as a vehicle/carrier for drug delivery. Without limitations, the composition can act as a scaffold to mimic the extracellular matrices (ECM) of the body, and/or promote tissue regeneration. The composition can serve both as physical support and/or adhesive.

As used herein, the term “wound” is used to describe skin wounds as well as tissue wounds. A skin wound is defined herein as a break in the continuity of skin tissue that is caused by direct injury to the skin. Several classes including punctures, incisions, excisions, lacerations, abrasions, atrophic skin, or necrotic wounds and burns generally characterize skin wounds. The compositions and methods of the invention are useful for enhancing the healing of all wounds of the skin. In particular, the present invention provides methods and compositions suitable for treatment of wounds in diabetics, normal patients and surgical patients. A “tissue wound” as used herein is a wound to an internal organ, such as a blood vessel, intestine, colon, etc. The materials of the invention are useful for enhancing the wound healing process in tissue wounds whether they arise naturally or as the result of surgery. For instance, during the repair of arteries the vessel needs to be sealed and wound healing must be promoted as quickly as possible. The compositions of the invention can speed up that process. The compositions of the invention are also particularly useful for the treatment of damaged tissue in the colon.

In some embodiments, the silk/platelet composition can be used to fill, volumize, and/or regenerate a tissue in need thereof. The silk/platelet compositions can generally be used for tissue filling or volumizing, soft tissue augmentation, replacement, cosmetic enhancement and/or tissue repair in a subject. Additionally, the compositions can be used for filling of any tissue void or indentation that are either naturally formed (e.g., aging) or created by surgical procedure for removal of tissue (e.g., a dermal cyst or a solid tumor), corticosteroid treatment, immunologic reaction resulting in lipoatrophy, tissue damage resulting from impact injuries or therapeutic treatment (e.g., radiotherapy or chemotherapy). The silk/platelet compositions can also be used to raise scar depressions.

In certain embodiments, the silk/platelet compositions can be used for soft tissue augmentation. As used herein, by the term “augmenting” or “augmentation” is meant increasing, filling in, restoring, enhancing or replacing a tissue. In some embodiments, the tissue can lose its elasticity, firmness, shape and/or volume. In some embodiments, the tissue can be partially or completely lost (e.g., removal of a tissue) or damaged. In those embodiments, the term “augmenting” or “augmentation” can also refer to decreasing, reducing or alleviating at least one symptom or defect in a tissue (for example, but not limited to, loss of elasticity, firmness, shape and/or volume in a tissue; presence of a void or an indentation in a tissue; loss of function in a tissue) by injecting into the tissue with at least one injectable composition described herein. In such embodiments, at least one symptom or defect in a tissue can be decreased, reduced or alleviated by at least about 30%, at least about 40%, at least about 50%, at least about 60%, at least about 70%, at least about 80% or higher, as compared to no treatment. In some embodiments, at least one symptom or defect in a tissue can be decreased, reduced or alleviated by at least about 90%, at least about 95%, at least about 97%, or higher, as compared to no treatment. In some embodiments, at least one symptom or defect in a tissue can be decreased, reduced or alleviated by 100% (defect-free or the defect is undetectable by one of skill in the art), as compared to no treatment. In other embodiments, the tissue can be augmented to prevent or delay the onset of defect manifestation in a tissue, e.g., loss of elasticity, firmness, shape and/or volume in a tissue, or signs of wrinkles. As used herein, the phrase “soft tissue augmentation” is generally used in reference to altering a soft tissue structure, including but not limited to, increasing, filling in, restoring, enhancing or replacing a tissue, e.g., to improve the cosmetic or aesthetic appearance of the soft tissue. For example, breast augmentation (also known as breast enlargement, mammoplasty enlargement, augmentation mammoplasty) alters the size and shape of a woman's breasts to improve the cosmetic or aesthetic appearance of the woman. Examples of soft tissue augmentation includes, but is not limited to, dermal tissue augmentation; filling of lines, folds, wrinkles, minor facial depressions, and cleft lips, especially in the face and neck; correction of minor deformities due to aging or disease, including in the hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; bulking agent for periurethral support, filling of indentations in the soft tissue, dermal or subcutaneous, due to, e.g., overzealous liposuction or other trauma; filling of acne or traumatic scars; filling of nasolabial lines, nasoglabellar lines and intraoral lines. In some embodiments, the silk/platelet compositions and/or silk fibroin particles described herein can be used to treat facial lipodystrophies. In some embodiments, the silk/platelet compositions can be used for breast augmentation and/or reconstruction.

In some embodiments, the silk/platelet compositions can be used for soft tissue repair. The term “repair” or “repairing” as used herein, with respect to a tissue, refers to any correction, reinforcement, reconditioning, remedy, regenerating, filling of a tissue that restores volume, shape and/or function of the tissue. In some embodiments “repair” includes full repair and partial repair. For example, the volume, shape and/or function of a tissue to be repaired can be restored by at least about 30%, at least about 40%, at least about 50%, at least about 60%, at least about 70%, at least about 80% or higher, as compared to no treatment. In some embodiments, the volume, shape and/or function of a tissue to be repaired can be restored by at least about 90%, at least about 95%, at least about 97%, or higher, as compared to no treatment. In some embodiments, the volume, shape and/or function of a tissue to be repaired can be restored by 100% (defect-free or the defect is undetectable by one of skill in the art), as compared to no treatment. In various embodiments, the silk/platelet compositions can be used to repair any soft tissues discussed earlier, e.g., breast, skin, and any soft tissues amenable for soft tissue augmentation. In some embodiments, the term “repair” or “repairing” are used herein interchangeably with the term “regeneration” or “regenerate” when used in reference to tissue treatment.

In some embodiments, the silk/platelet compositions can be used for soft tissue reconstruction. As used herein, the phrase “soft tissue reconstruction” refers to rebuilding a soft tissue structure that was severely damaged or lost, e.g., by a dramatic accident or surgical removal. For example, breast reconstruction is the rebuilding of a breast, usually in women. Conventional methods of construct a natural-looking breast generally involve using autologous tissue or prosthetic material. In some embodiments, such breast reconstruction can include reformation of a natural-looking areola and nipple, wherein such procedure can involve the use of implants or relocated flaps of the patient's own tissue. In some embodiments, administration of silk/platelet compositions and/or silk fibroin particles into a soft tissue region to be reconstructed can maintain the shape and/or size of the reconstructed soft tissue structure for a period of time, e.g., at least 6 weeks, at least about 2 months, at least about 3 months or longer.

Without wishing to be bound, some embodiments of the silk/platelet compositions can be used for hard tissue (musculoskeletal) augmentation or repair, such as augmentation or repair of bone, cartilage and ligament.

The silk/platelet compositions and silk fibroin particles described herein can also be used for filling a tissue located at or near a prosthetic implant, for example, but not limited to, a conventional breast implant or knee replacement implant. In some embodiments, the silk/platelet compositions and silk fibroin particles can be used to interface between a prosthetic implant and a tissue, e.g., to fill a void between the prosthetic implant and the tissue, and/or to prevent the tissue in direct contact with the prosthetic implant. By way of example only, after placing a prosthetic implant (e.g., a breast implant) in a subject, an injectable composition described herein can be introduced at or adjacent to the implant to fill any void between the implant and the tissue (e.g., breast tissue) and/or “sculpt” the tissue for a more natural look

In any of the uses described herein, silk fibroin particles could be combined with cells for purposes of a biologically enhanced repair. Cells could be collected from a multitude of hosts including but not limited to human autograft tissues, or transgenic mammals. More specifically, human cells used can comprise cells selected from stem cells (e.g., adipocyte-derived stem cells), osteocytes, fibroblasts, lipocytes, assorted immunocytes, cells from lipoaspirate or any combinations thereof. In some embodiments, the cells can be added into the silk/platelet composition, carrier solution, or mixture of silk/platelet composition and carrier solution prior to administration.

After preparation, the compositions described herein can be sterilized using conventional sterilization process such as radiation-based sterilization (i.e. gamma-ray), chemical based sterilization (ethylene oxide), autoclaving, or other appropriate procedures. In some embodiments, sterilization process can be with ethylene oxide at a temperature between from about 52° C. to about 55° C. for a time of 8 or less hours. The composition described herein can also be processed aseptically. Sterile silk/platelet compositions described herein can be packaged in an appropriate sterilize moisture resistant package for shipment.

In another aspect, the compositions disclosed herein can be uses in methods of promoting wound healing or wound closure, for example, at an incision site. The methods generally comprise implanting, administering, or placing a silk/platelet composition as disclosed herein at the wound or incision site. In some embodiments, the compositions disclosed herein can be used in methods of soft tissue repair or augmentation. The methods generally comprise implanting, administering, or placing a silk/platelet composition as disclosed herein at the desired site.

Without limitations, the compositions disclosed herein can be used in personalized medicine. For example, a subject's own blood can be used for the platelets to be included in the composition. Thus, the compositions can be applied safely on a person-by-person basis. In emergency applications, the compositions can be administered to a person in need by using blood from another person or a group of people.

Without wishing to be bound by a theory, the silk/platelet composition described herein provides a number of advantages, such as:
1. Tunable mechanical properties of the silk phase to influence the silk/platelet PG
2. Tunable rates of degradation of the silk phase to influence the silk/PG
3. Synergies in double-network mechanical properties by controlled gelation of PG and silk
4. Tunable release of growth factors via modification of the silk phase by varying) silk solution concentration, ii) boiling time, iii) sterilization method (filter vs. autoclave).
5. Sustained release of growth factors to modulate tissue repairs and provide improved healing and regeneration.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The term “herein” is meant to include all of the disclosure and is not intended to be limited to a subsection of the disclosure.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein, the term “microparticle” refers to a particle having a particle size of about 0.01 μm to about 1000 μm.

As used herein, the term “nanoparticle” refers to particle having a particle size of about 0.1 nm to about 1000 nm.

It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

As used herein, a “subject” means a human or animal. Usually the animal is a vertebrate such as, but not limited to a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. Additionally, a subject can be an infant or a child.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with autoimmune disease or inflammation. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. . . . . In some embodiments, the subject can be a patient or other subject in a clinical setting. In some embodiments, the subject can already be undergoing treatment.

The disclosure is further illustrated by the following examples, which should not be construed as limiting. The examples are illustrative only, and are not intended to limit, in any manner, any of the aspects described herein. The following examples do not in any way limit the invention.

### EXAMPLES

### Example 1

### Silk Hydrogels Used for Platelet Gel Augmentation and Controlled Release of Encapsulate

### Production and Characterization of Silk/Platelet Gels

Materials:

Human peripheral blood was purchased from Research Blood Component, LLC (Boston, Mass.). Calcium gluconate was purchased from APP Pharmaceuticals (Lake Zurich, Ill.). Bombyx mori silkworm cocoons were supplied by Tajima Shoji Co., LTD (Yokohama, Japan). Histology reagents including ε poly-L-lysine and Masson's Trichrome were purchased from Sigma Aldrich (St. Louis, Mo.). Silk filtration and purification was conducted using dialysis tubing from Spectrum Laboratories Inc. (Rancho Dominguez, Calif.) and centrifugal filter units from Millipore (Billerica, Mass.). The CellTiter Cell Proliferation Assay (MTS) was purchased from Promega (Madison, Wis.). Cell proliferation assays were conducted using human umbilical vein endothelial cells (HUVECs; Cambrex, East Rutherford, N.J.) with control media (endothelial cell basal medium-2) and complete media (endothelial cell basal medium-2, hydrocortisone, hEGF, FBS, VEGF, hFGF-B, R3-IGF-1, ascorbic acid, heparin). All cell culture media components were obtained from Lonza (Basel, Switzerland) and used at standard concentrations. The following antibodies have been used: mouse anti-posphoERK, mouse anti-actin from Cell Signaling (Danvers, Mass.), rabbit anti-VE-cadherin from LifeSpan Bioscences (Seattle, Wash.) and rabbit anti-CD31 from Abcam (Cambridge, Mass.). UO 126 was from Calbiochem (San Diego, Calif.). Human VEGF, TGF-β1, PDGF-AB DuoSet and human VEGF affinity purified polyclonal antibody were purchased from R&D system (Minneapolis, Minn.). Recombinant-VEGF-165 was purchased from Shenandoah Biotechnology Inc. (Warwick, Pa.).

Platelet Gel (PG) Preparation:

Human platelets were obtained from healthy volunteers in citric acid/citrate/dextrose solution. Whole blood was centrifuged at 120×g for 15 minutes to obtain platelet rich-plasma. PRP was subsequently centrifuged at 100×g for 15 minutes to eliminate leukocytes in the supernatant. Platelets were recovered by an additional centrifugation at 720×g for 15 minutes to obtain a pellet of platelets and a supernatant of plasma poor of platelets (PPP). Platelet count was adjusted to a final concentration of 4 x 106 platelets/μL by re-suspending platelets in PPP. Autologous thrombin was prepared by mixing (5:1, v/v) PPP with 0.22 M calcium gluconate (Mazzucco et al., Transfusion 2004, 44, 1013). After 15 minutes incubation at 37° C. and centrifugation at 1000×g for 15 minutes, the thrombin-containing supernatant was collected. PGs were obtained by mixing PRP (final concentration 2 x 106 platelets/μL)/autologous thrombin/calcium gluconate 0.22 M (ratio 8:2:1) and incubated in a humidified chamber at 37° C. until use. To evaluate growth factor release from PG, 500 μL of PBS was added to each sample after gelation, which occurred in about 20 minutes. Separately, plasma enriched in growth factors was formed by removal of the fibrin gel, diluted 1:1 with phosphate buffered saline (PBS; Invitrogen) or 1% w/v silk solution. For these growth factor studies, the samples were stored at 37° C., or room temperature or 4° C.

Silk Gel Preparation:

Silk fibroin aqueous solution was obtained from B. mori silkworm cocoons using previously described procedures (Kim et al. Biomaterials 2005, 26, 2775). The boiling time was modified from 30-60 minutes in certain experiments, as indicated, in order to modify the molecular weight of the silk solution as previously described (Wray et al., J. Biomed. Mater. Res. B Appl. Biomater. 2011, 99, 89). The resulting 6-8% (w/v) fibroin solution was diluted in ultra pure water to obtain a 4% (w/v) silk solution, or concentrated by placing the solution in the dialysis cassettes and letting the excess of water evaporate at RT for periods of time depending on the desired concentration. Solutions were then sterilized by 0.2 μm filtration (Millipore) or by standard 20 minute liquid autoclave cycle, as previously reported to sterilize solutions prior to sonication (Wang et al., Biomaterials 2008, 29, 1054). The resultant solutions were sonicated at different amplitudes and time with a Branson 450 Sonifier (Branson Ultrasonics Co., Danbury, Conn.) (Wang et al., Biomaterials 2008, 29, 1054). In order to investigate the impact of the charges in growth factor release, 10% w/v ε-poly-L-lysine or 0.1% w/v silk fibroin ionomers (silk fibroin-poly-L-lysine and silk fibroin-poly-L-glutamic acid ionomers) (Calabrese, Kaplan, Biomaterials 2012, 33, 7375) were added to the 4% silk solution prior to the sonication. The sonicated silk solution, cooled to room temperature in ice for 1 minute, was subsequently mixed in a ratio 1:1 with PRP, generating PG-Silk. A volume of 500 μL, of the solution was incubated in 24 well plates in an incubator at 37° C. to promote gelation. A volume of 500 μL of PBS was then added to each sample, after gelation was visually confirmed. At different time points (1 hrs, 1 day, 7 days, 14 days, 21 days) the PBS from each sample was collected and frozen at −80° C., and completely replaced with fresh PBS.

At the last time point of incubation, the silk/platelet gel samples (500 μL/well) were solubilized in 500 μL, urea 8M for 30 minutes at 37° C. Residual salt was removed by dialysis against deionized water (by using dialysis tubing with MWCO 2 kDa) for 16 hrs. The obtained solution was concentrated to a final volume of 500 μL, by using centrifugal filter units (3 kDa cut-off).

In some experiments, PG-Silk samples mixed with silk fibroin ionomers were analyzed for their ability to swell. The weight of the samples was recorded 1 hr after preparation. After adding 500 μL, of PBS, samples were allowed to swell for 24 hrs and measured again, and the percentage weight gain was calculated.

Growth Factor Evaluation:

PG-silk was evaluated for growth factor release at time points of 1 hr, 1 day, 7 days, 14 days, 21 days incubation. The platelet gels were removed from their 37° C. storage conditions, centrifuged at 2000×g for 15 minutes at RT in order to separate the gel and the PBS supernatant rich in growth factors, which was immediately stored at −80° C. At the final 21 day collection, the PG-Silk samples were solubilized. The collected growth factor solutions were quantified through enzyme-linked immunosorbent assay (ELISA) kits for platelet-derived growth factor-AB (PDGF-AB), transforming growth factor-beta1 (TGF-β1) and vascular endothelial growth factor (VEGF) according to manufacturer's instructions.

Rheometer Testing:

In order to monitor the gelation kinetics and capture the rheological shear stiffness of the various PG-Silk formulations, several designated samples of silk solution diluted in water, or PPP, or platelet gel (ratio 1:1) were prepared for rheology analysis and compared to platelet gel alone. Dynamic oscillatory time sweeps were performed using an ARES strain-controlled rheometer (TA Instruments, New Castle, Del.) with 25-mm-diameter stainless steel cone-and-plate geometry (1° cone angle) at 0.051-mm measuring gap distance. In a typical experiment, the silk solution was applied slowly via pipette on the rheometer plate to prevent shearing of the sample immediately after sonication. The normal force applied on the sample during lowering of the top plate was limited to 20 grams. A low viscosity mineral oil was used to prevent sample evaporation from the sides of the plate, as previously described (Yucel et al. Biophys. J. 2009, 97, 2044). An air-driven oven was used to control temperature at 37° C. for the duration of the test. Dynamic oscillatory time sweeps were collected at a low strain amplitude (γ=1%, w=3.14 rad/s) to prevent possible sample damage due to applied shear during measurements. This testing protocol was applied continuously over 1 hour for PG alone, which decayed quickly after reaching its maximum, and 16 hours for all silk containing groups, which asymptotically approached a maximum threshold over that time span.

Compression Testing:

For unconfined compression testing, silk solutions were generated from cocoons boiled from 30- to 60-minute and all solutions with concentration ranging from 4 w/v % to 12 w/v %. Where possible, sterilization via filtration or autoclave cycle was performed in advance of sonication. For all groups, a larger volume of sonicated silk (2 mL-3 mL) was mixed with either PPP or PG in a 1:1 ratio and cast into 30 cm petri dishes (Kluge et al., J. Mech. Behay. Biomed. Mater. 2010, 3, 278; Wang et al., Biomaterials 2008, 29, 1054). Plugs were prepared after gelation using a 6 mm inner diameter biopsy punch. The gel plugs were left in PBS prior to testing. A strain-to-failure test was used to extract an elastic modulus. A minimum of N=4 samples were evaluated and were tested on a 3366 Instron machine (Norwood, Mass.) equipped with unconfined compression platens and a 100 Newton (N) load transducer and sample data exported using Bluehill Software Version 2.0. Each sample was compressed at a strain rate of 0.1%/s, beginning after nominal tare loads (0.005N) were reached and sample heights recorded. The compressive stress and strain were determined by normalizing against sample geometries and the elastic modulus was calculated as the best fit linear regression established at a 5% strain range of each stress/strain curve (Kluge et al., J. Mech. Behay. Biomed. Mater. 2010, 3, 278; Wang et al., Biomaterials 2008, 29, 1054).

Cell Proliferation Assay:

HUVECs were seeded in 96 well plates at a density of 5000 cells/well, supplemented with control medium. One day later, media was fully replaced with complete medium in positive control groups, control medium containing 25% (v/v) PG-Silk supernatant in experimental groups, or control medium alone in negative control groups. After 3 days, the MTS assay was performed by following the manufacturer's instructions. The absorbance was measured at 490 nm in a microplate reader (Molecular Devices). In order to evaluate molecular pathway activation, cells were prepared as described above and, one day after seeding, HUVECs were supplemented with control medium containing 25% (v/v) PG-Silk supernatant with 10 μM UO 126, a pharmacological inhibitor of ERK, or 10 μg/mL anti-VEGF blocking antibody. After 48 hrs the MTS assay was performed.

Western Immunoblotting:

After 2 hours of incubation with UO 126 or anti-VEGF blocking antibody, HUVECs were washed with PBS and lysed with RIPA buffer for 20 minutes in ice. The lysates were clarified by centrifugation at 15700×g at 4° C. for 15 minutes. Protein concentration was measured by the bicinchoninic acid assay (Pierce), according to the manufacturer's instructions. Samples containing equal amounts of proteins were subjected to SDS-polyacrylamide gel electrophoresis (SDS-PAGE) and then immunoblotted with antibodies against pERK (1:1000) or actin (1:1000). Immunoreactive bands were detected by a horseradish peroxidase-labeled secondary antibody, using an enhanced chemiluminescence reagent.

In Vivo Injection of PG-Silk Formulations:

Animals were cared for in compliance with Tufts University Institutional Animal Care and Use Committee (IACUC) in accordance with the Office of Laboratory Animal Welfare (OLAW) at the National Institutes of Health (NIH). Athymic nude rats (RNU, Charles River) were allowed to acclimate for 1 week prior to implantation and maintained in sterile housing. The animals were anesthetized using isoflurane (4% induction, 2.5% maintenance). Each animal received 6×300 μL subcutaneous injections of either PG-Silk (2%, 6% w/v, diluted in ultrapure water or PG at a 1:1 ratio) or PG alone in random distribution. Given prior experience with silk alone, a pilot study was first performed to see if any PG-containing groups would be dispersed in the subcutaneous tissue following injection or if they would stay local to the site of injection. For this pilot study, we injected all PG-containing formulations (N=2 each), with sacrifice at 12 hrs. Subsequent trials included all groups listed above (N=3 injections per material) with sacrifice at 2 and 4 week time points. Animals were visually analyzed daily for the first three days, and then three times a week, in order to monitor the sites of injection for changes in physical appearance.

Histology and Immunohistochemistry:

Harvested tissues were stored in formalin for 48 hrs and then processed through a series of alcohol dehydration solvents before paraffin embedding. Samples were cut in 10 μm sections for subsequent staining with Hematoxylin and Eosin (H&E) and Masson's Trichrome, and immunohistochemistry described below.

For immunohistochemistry, the sections were blocked with serum and incubated with anti-CD31 or -VE-cadherin. Sections were then washed in PBS, incubated with a secondary anti-rabbit antibody for 30 min and finally with ImmPACT DAB enzyme substrate for 5 min. After washing with water, the sections were counterstained with hematoxylin and mounted.

Statistics:

Values were expressed as mean±SD (standard deviation). Student's t-test was performed for paired observations. Multiple comparisons were evaluated by one-way analysis of variance (ANOVA) followed by Bonferroni's post hoc tests. A value of p<0.05 was considered statistically significant.

### Results of Characterization

Kinetic Profile of Growth Factors Released by PG-Silk:

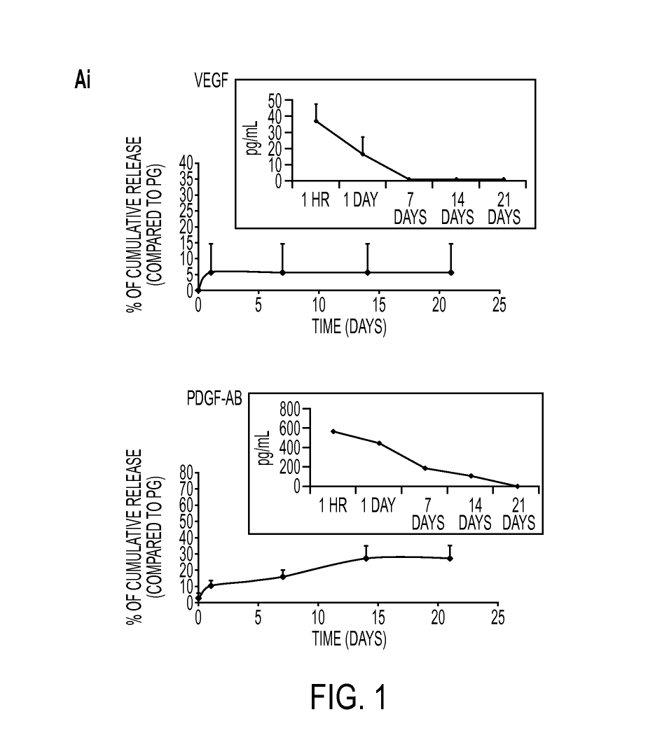

PG-Silk was evaluated for their ability to sustain platelet growth factor release. The kinetic profiles of VEGF, PDGF-AB and TGF-β1 were analyzed by ELISA overtime (21 days) and compared to growth factors released by PG. In PGs, as indicated in the insert of each graph (FIG. 1Ai), VEGF, PDGF-AB and TGF-01 were released promptly into the PBS supernatant in 1 hour, with a progressively-decreasing release overtime. In the case of growth factors released from silk-PGs, VEGF and TGF-β1 were detected at very low levels during the 21-day release study as compared to PG groups. Moreover, while VEGF and PDGF-AB were released at higher levels at earlier time points, with PDGF-AB progressively increasing overtime, TGF-β1 was detected only at the last time point (21 day).

In order to differentiate between growth factors degraded while entrapped in silk vs. the non-specific binding or entrapment of the factors within the silk matrix (as it has been previously observed for antibodies entrapped in silk) (Guziewicz et al., Biomaterials 2011, 32, 2642). PG-silks were solubilized at day 21 using urea 8M, a process which required 30 minutes for completion, as it could be visually confirmed. Following PG-Silk solubilization and dialysis to desalt the mixture, the recovered material was concentrated, and then evaluated by ELISA for the presence of VEGF, TGF-β1 and PDGF-AB. While VEGF and PDGF-AB were not detected in the solubilized PG-Silks, TGF-β1 was detected at a high level (3091.4±441.4 pg/mL). In FIG. 1Aii we show that the % of TGFβ-1 recovered from the solubilized PG-Silk complex with urea was about 200% as compared to TGFβ-1 released from PG into PBS at 1 hr (i.e. the maximum theoretical TGFβ-1 which could be released by activated platelets into PBS from within the PG clot).

We hypothesize that the increased amount of TGFβ-1 obtained from PG-Silks may be ascribed to the ability of urea to dissolve the clot, which potentially entraps a large amount of growth factors compared to non-associated factors in plasma. Finally, preliminary experiments performed with recombinant VEGF (rVEGF) showed that the presence of urea 8M did not affect the rVEGF recovery, however almost the 50% was still retained in silk (FIG. 1Aiii). These results suggest that increased growth factor stores were originally available in the PG-Silk systems but were partially diminished in our experiments by urea treatment—these stores would otherwise be available over time by degradation-mediated release from the silk gel.

Silk Properties in Modulating Growth Factor Release:

Considering the net charge at pH 7.4 of the growth factors analyzed (2.2 VEGF, 14.3 PDGF-AB, 8.4 TGF-β1), and of silk (−36.1), we investigated the involvement of electrostatic interactions in growth factor release from silk. The silk solution was mixed with 0.1% w/v silk fibroin ionomers (silk-poly-L-lysine or silk-poly-L-glutamate), prior to induce gelation through sonication and mix with PRP-calcium gluconate and autologous thrombin. VEGF and PDGF-AB were analyzed by ELISA over 21 days (FIG. 1Bi). Because of the interference in the ELISA assay of the silk ionomers with the acidic-basic procedure used to activate the latent form of TGFβ-1 (data not shown), the contribution of silk ionomers in TGFβ-1 release was not analyzed. The positively-charged ionomer silk-poly-L-lysine promoted the release of both VEGF and PDGF-AB into the PBS promptly in 1 hr, as compared to unmodified silk, suggesting a contribution of the electrostatic forces in growth factor release. On the contrary, during the early time points, the negatively charged silk-poly-L-glutamate ionomer-PG system released growth factors at the same rate as unmodified silk. However, silk-poly-L-glutamate promoted a sustained growth factor release at the late time points, as compared to unmodified silk. We observed similar changes in growth factor release within silk by addition of soluble poly-L-lysine prior to gelation by sonication/PG activation, confirming that the data linked to charge modification was not due solely to the means of silk modification (data not shown).

To confirm that these effects were related specifically to the charge, the net charge of silk and silk-ionomers was analyzed by zeta potential measurements. Even when diluted in plasma and calcium gluconate, following the procedure used to analyze the growth factor release, the silk maintained an overall negative charge similar to the silk diluted in water, that was increased when mixed with poly-L-glutamate, and decreased when mixed with poly-L-lysine (Table 1). The swelling of silk gel, known to be a critical step in drug release (Guziewicz et al., Biomaterials 2011, 32, 2642; Pritchard et al., 2011, Expert Opin. Drug Deliv. 2011, 8, 797), is expected to be greater in the presence of charges than in unmodified silk, due to the high charge density conferring higher hydrophilicity (Guziewicz et al., Biomaterials 2013). To this regard, the swelling behavior of PG-Silk mixed with silk ionomers was compared to that of PG-Silk alone. An increased swelling, in 24 hrs, was observed both for silk mixed with poly-L-lysine and poly-L-glutamate, as compared to silk alone (FIG. 1Bii). All together these data indicate that the growth factor release is modulated by changing the net charge of silk, through the use of silk fibroin ionomers.

TABLE 1
Charge of 2% silk and silk mixed with silk fibroin ionomers, diluted
in water or plasma, determined by zeta potential measurements.

| Zeta Potential | | | |
|---|---|---|---|
| (mV) | Silk | Silk-poly-L-lysine | Silk-poly-L-glutamate |
| In water | −7.25 ± 1.11 | 0.297 ± 0.11 | −10.04 ± 1.05 |
| In plasma | −4.81 ± 0.07 | −0.93 ± 0.23 | −5.93 ± 0.7 |

Moreover, ionomers are able to modulate the release of growth factors by exerting a synergistic effect: through the modulation of the electrostatic interactions, resulting in an increased release promoted by the cationic ionomer silk-poly-L-lysine, and through the increased swelling potential compared to silk alone. This swelling effect amplifies the charge effect in the case of the silk-poly-L-lysine, and overcomes the retention of the growth factors exerted by silk-poly-L-glutamate in the late time points, where the effect of the charges is no longer predominant because of the dilution effect.

VEGF and pERK Signaling in HUVEC Proliferation Induced by PG-Silk:

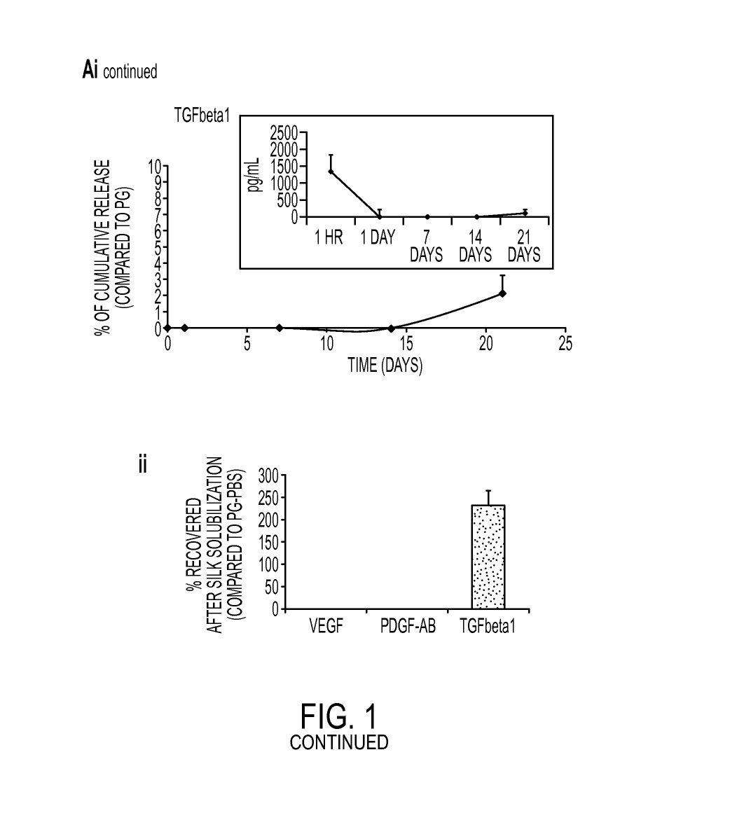

To investigate if growth factors released by PG-Silks were biologically active, we evaluated their effects on HUVEC proliferation. We tested the PG-Silk supernatants collected at 1 hr, 1 day, 7, 14 and 21 days. The MTS assay revealed that control medium supplemented with PG-Silk supernatants collected at each time point (25%, taken from PBS added to the PG-Silk well), was able to promote HUVEC proliferation, as compared to control medium alone (p<0.05) (FIG. 2 A).

VEGF, through its receptor kinase insert domain-containing receptor (KDR), is known to be a strong activator of ERK 1 and 2, resulting in HUVEC proliferation. To analyze the ability of VEGF released by PG-Silk to mediate properly the same outside-in signaling pathway, ERK phosphorylation in HUVECs was analyzed. PG-Silk supernatant collected at day 7 was able to induce ERK1/2 phosphorylation greater than expansion media, as revealed by western blotting analysis (FIG. 2B). To confirm the involvement of VEGF mediated-ERK signaling induced by PG-Silk, HUVECs were preincubated with a pharmacological inhibitor of ERK, UO 126. FIG. 2C shows that HUVEC proliferation, induced by PG-Silk collected at day 7, was attenuated when ERK was inhibited. Moreover, depletion of VEGF from PG-Silk supernatant through a neutralizing antibody against VEGF, resulted in a decreased HUVEC proliferation, suggesting that VEGF released by PG-Silk contributes at least partially to HUVEC proliferation. A concomitant decrease in ERK phosphorilation in HUVEC exposed to the neutralizing antibody against VEGF was demonstrated by western blotting (FIG. 2D).

Silk Properties in Platelet Growth Factors Stabilization:

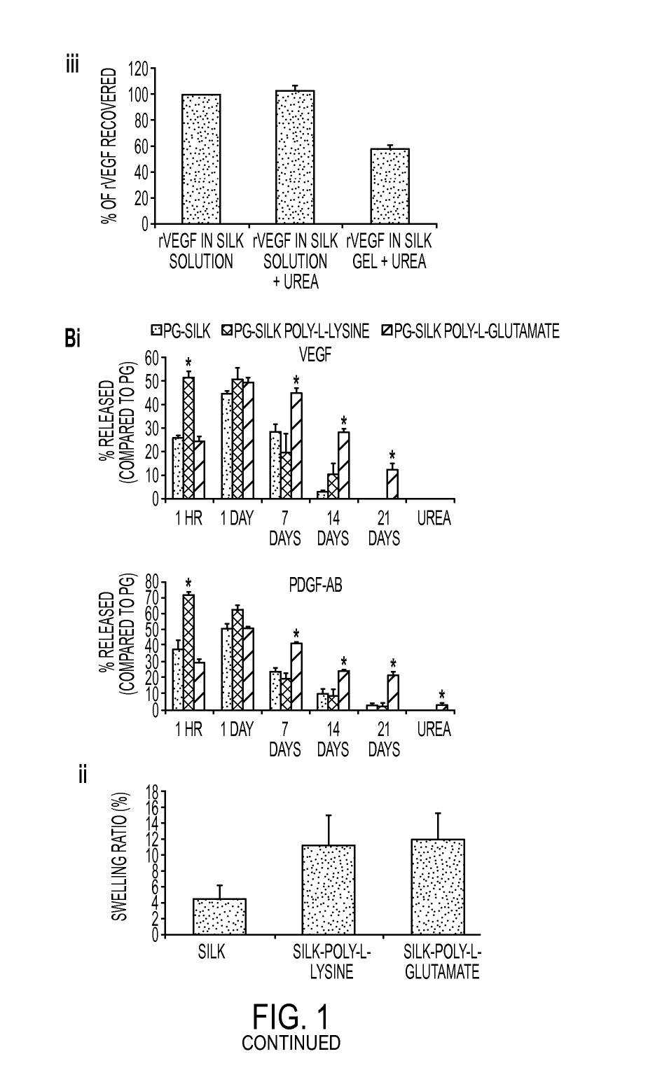

The ability of silk to stabilize the platelet-derived growth factors was investigated. To assess this hypothesis, the recovery of rVEGF overtime was analyzed. When rVEGF was loaded to a 2% w/v silk solution, its concentration (measured by ELISA) was constant over 16 days (FIG. 3A). On the contrary, rVEGF, when diluted in PBS (or water, data not shown), was almost completely degraded in 2 days, as expected. Based on this evidence, we analyzed the ability of silk to stabilize the growth factors released by PG-Silk. The silk solution, after sonication, was mixed with PG. The resulting gel was re-suspended in 1% silk solution or PBS overtime. While silk did not show a stabilizing effect on PDGF-AB (known to be stable also in plasma (data not shown)), it was able to stabilize TGFβ-1 over 7 days (FIG. 3 B). Moreover, when the growth factors obtained from PG were diluted 1:1 in 1% silk solution, a significantly higher concentration of TGF-β1 was detected, by ELISA assay, after 2 weeks, as compared to growth factors diluted 1:1 in PBS (FIG. 3C). This increase in detectable TGF-β1 occurred during samples storage at 37° C., as well as at room temperature and 4° C. These experiments suggest that silk is able to stabilize TGF-β1 greater than plasma, and this mechanism is temperature independent. All together these findings suggest that the silk can have a stabilizing effect on released growth factors, in solution phase, before the gelation event has finalized.

Rheological Silk Gelation Behavior:

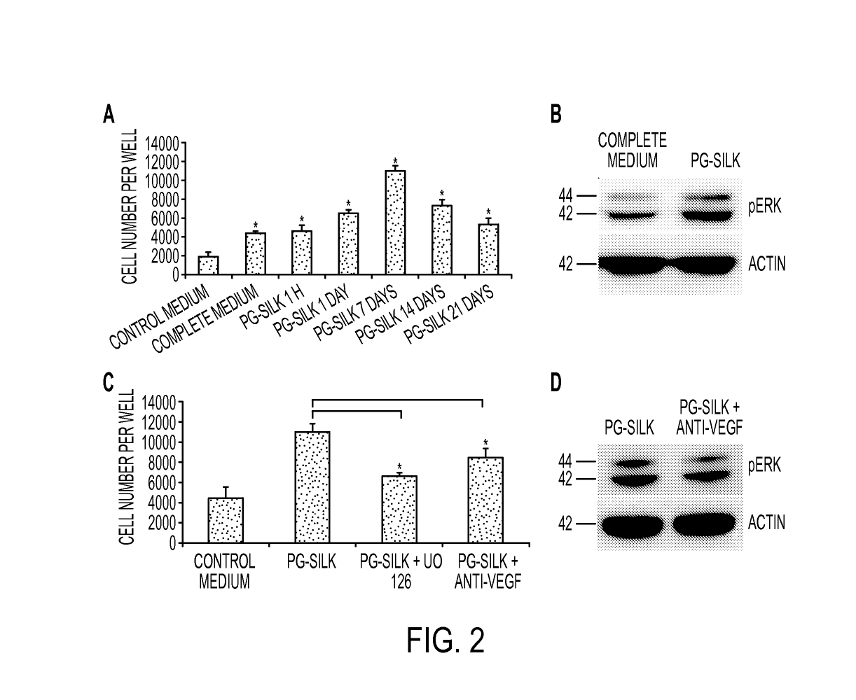

The stiffness of silk fibroin hydrogels can be tuned by controlling the solution concentration, and this has been most heavily quantified in terms of compressive properties (Kluge et al., J. Mech. Behay. Biomed. Mater. 2010, 3, 278; Wang et al., Biomaterials 2008, 29, 1054). To confirm that rheological stiffness of PG-Silk was dependent on incorporation of silk and its concentration, we prepared a range of concentrations of silk and induced gelation of both components immediately prior to initiating a series of nondestructive time-lapse dynamic oscillatory shear tests, as shown in FIG. 4A. The equilibrium stiffness values proved to be concentration-dependent as was shown previously for vortex-induced gels (Yucel et al., Biophys. J. 2009, 97, 2044). Interestingly, we routinely observed that the PG alone rapidly formed a fibrin clot, as denoted by the storage modulus peak located ˜480 seconds (—8 minutes). Following this peak, the storage modulus of PG precipitously decreased, and this was likely due to the fragile nature of the fibrin gel network and its propensity to dissociate from the plasma. Conversely, silk hydrogels continued to increase in storage modulus during oscillatory shear, and well beyond its initial point of increase until reaching a plateau at ˜16 hrs. This behavior is due to the relatively slow maturation of the silk gel physical crosslink network under sonication parameters employed here. Using these unique gelation signatures for the two protein components we were also able to resolve the PG gelation event in the context of a 1:1 dilution with sonicated silk gel (FIG. 4B,C), allowing us to verify that the presence of the silk gel did not inhibit normal platelet gel formation. Moreover, despite the relatively soft behavior of the PG alone compared to silk at 2% w/v concentration, each 2% silk gel reinforced with a PG network was significantly stiffer than its PPP-laden counterpart (FIG. 4B,C). These findings suggest that not only does the inclusion of added protein (in the case of PPP) contribute to the PG-Silk system's stiffness, but also its ability to form a fibrin gel network in the context of a more slowly forming silk gel.

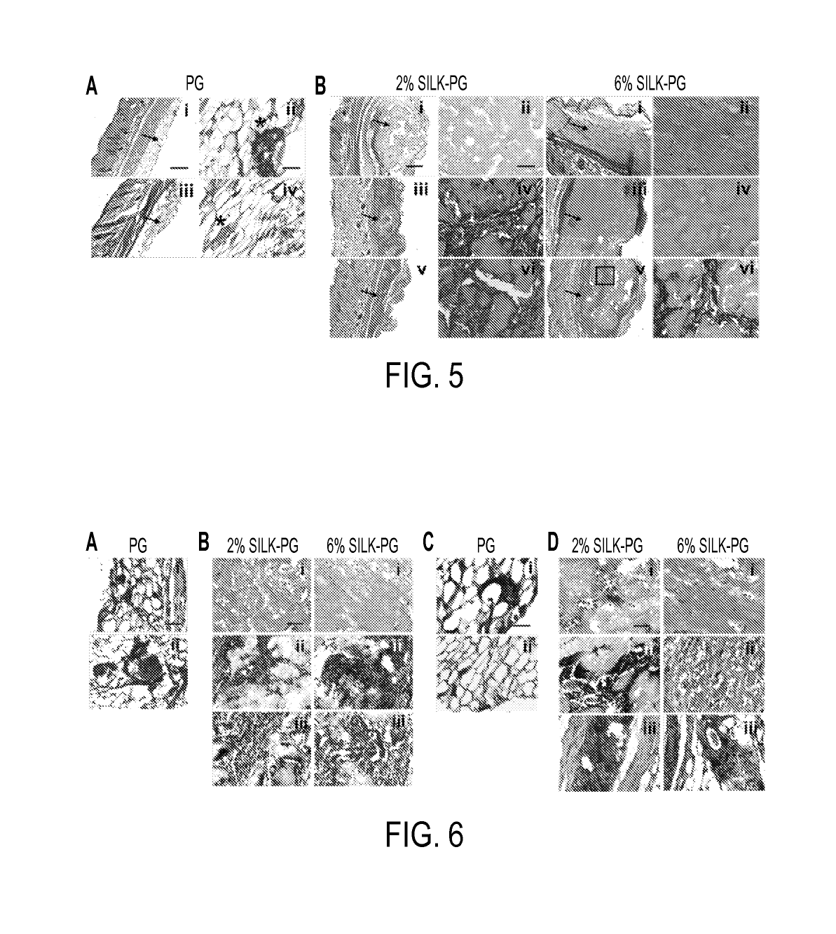

Since the various silk solution sterilization methods required prior to sonication and injection could affect the properties of the solution, and therefore the quality of the resultant hydrogel networks, we repeated these rheological assessments with variably-sterilized precursor silk solutions mixed with platelet gel components to draw comparisons to the virgin state of silk (i.e. the untreated solutions). Data comparing the two sterilization methods to the virgin silk solution conditions revealed no differences in rheological stiffness behaviors of the undiluted silk at 2% w/v nor the ability to participate in dual gel network formation (FIG. 7A). We also sought to confirm our rheological results with quasi-static compressive tests across a wider range of silk concentrations (FIG. 4D). We were able to concentrate solutions taken from cocoons boiled for 30 minutes and subsequently sterilize them by either 0.2 μm filtration or 20-minute autoclave cycle at up to 8 w/v % (prior to 1:1 dilution to 4 w/v % with either PPP or PG), but were unable to filter sterilize them at 10 w/v %. However, when solutions were generated from 45-minute- and 60-minute-boiled (hereafter referred to as 45 MB and 60 MB) silk cocoons, they could be successfully sterilized at higher concentrations using both methods (FIG. 7B). Boil time of silk solution did not significantly alter the compressive properties of the material across all concentrations, independent of PG inclusion vs. PPP, and this was consistent until the maximum concentration that could be sterile-filtered (i.e. 60 MB group at 14w/v % starting silk concentration).

In Vivo Analysis of PG, Silk Gel and PG-Silk Gel Behavior:

Nude rats were injected with the same volume of PG or 2% or 6% silk solution (with or without PG dilution) upon sonication in order to induce gelation after injection, and the in vivo degradation rate of the injected gels and cell infiltration were analyzed. No acute or chronic adverse reactions were observed in any animal group over time. The raised bumps from the injected bolus were stable for all groups immediately following injection. After 12 hrs the injection sites containing PG alone were no longer raised or palpable. However, the 2% silk groups, with and without PG, were visible overtime with a progressively decreasing size starting after 2 weeks from injection. Meanwhile, the 6% silk groups, with and without PG, were visible over 1 month and continuously maintained the same size throughout the study.

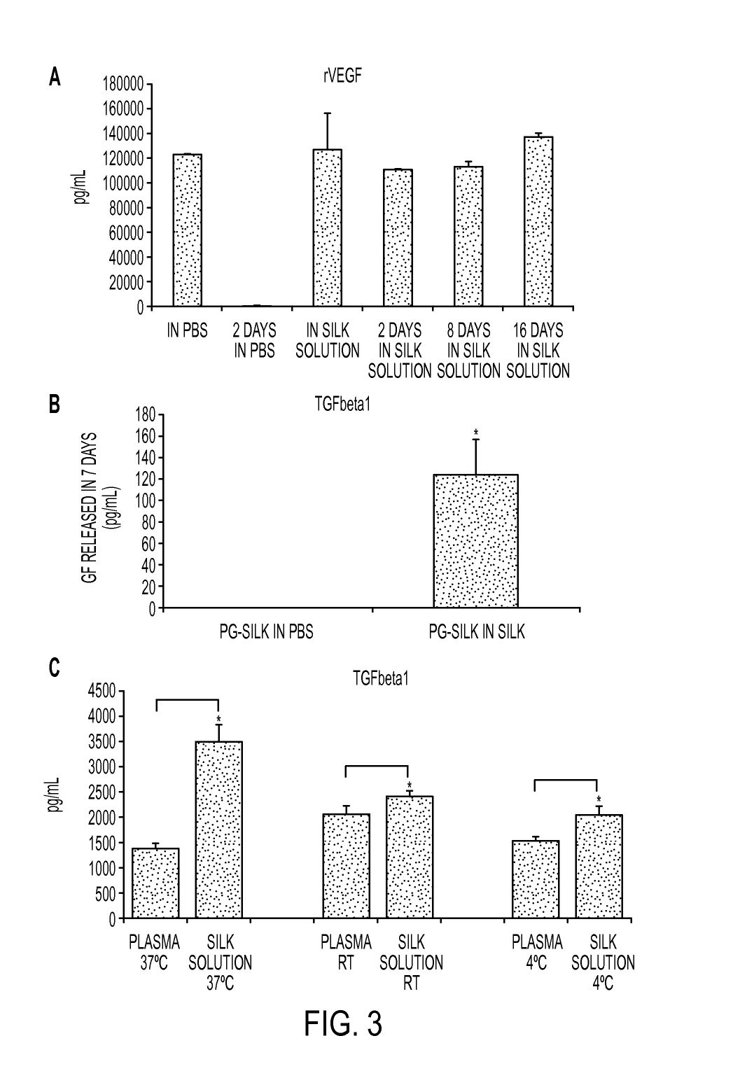

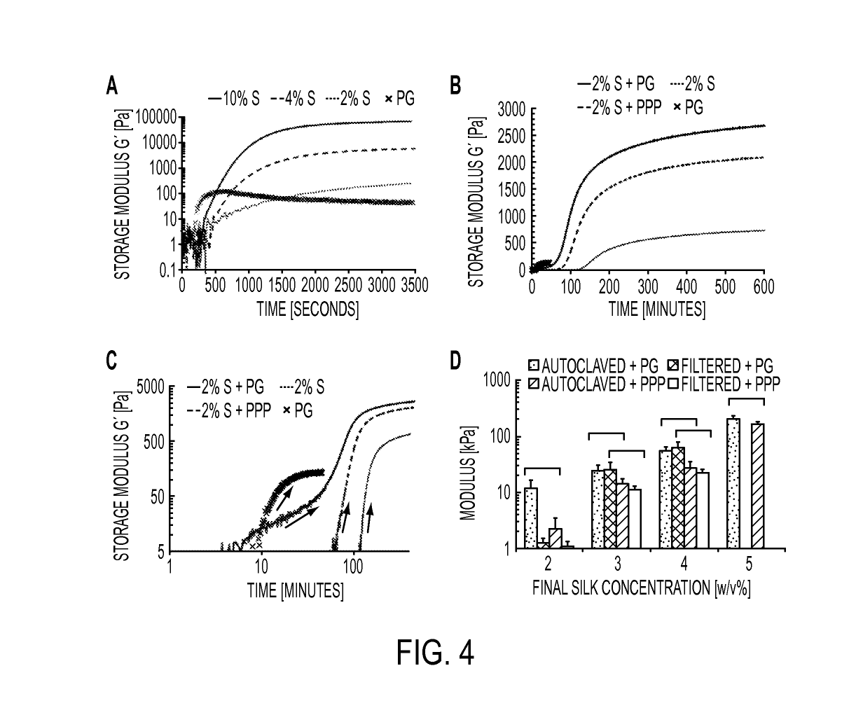

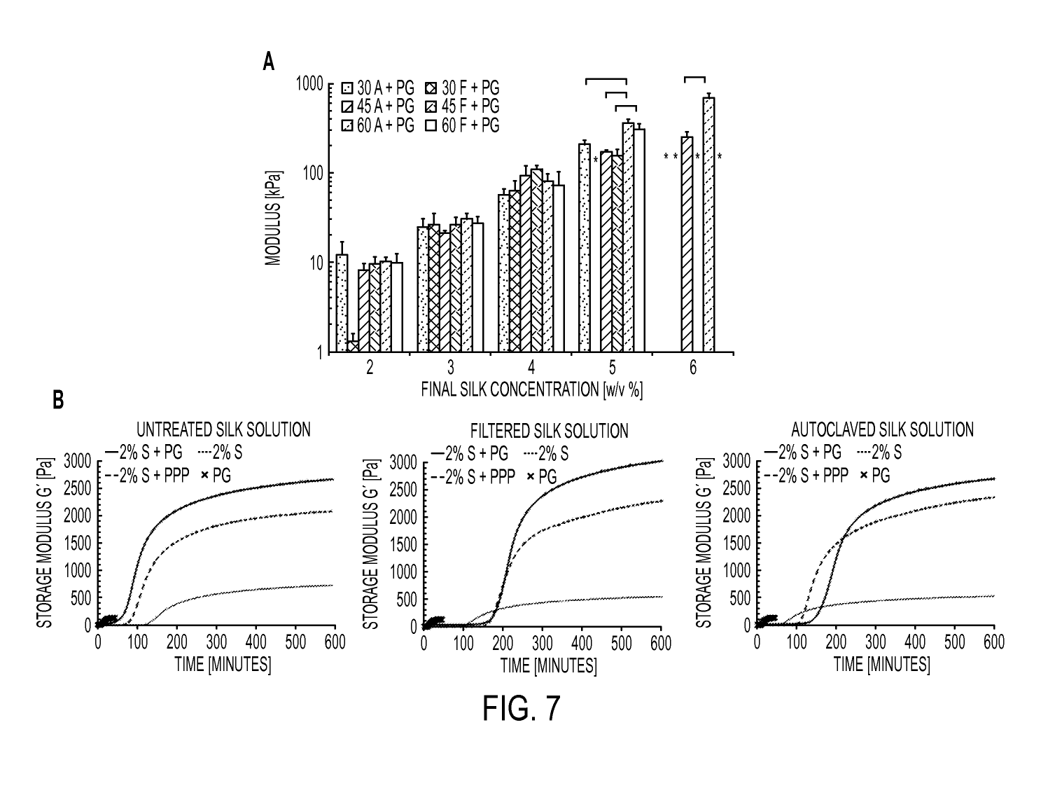

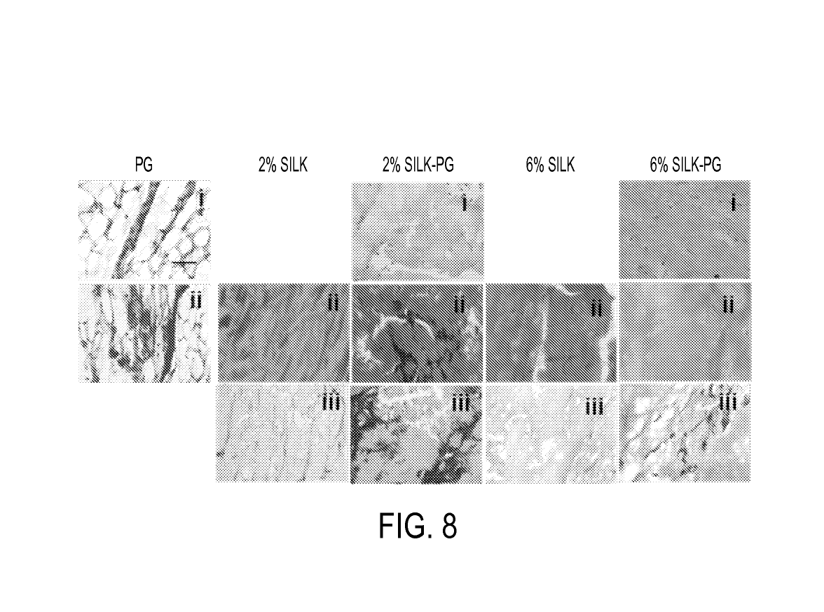

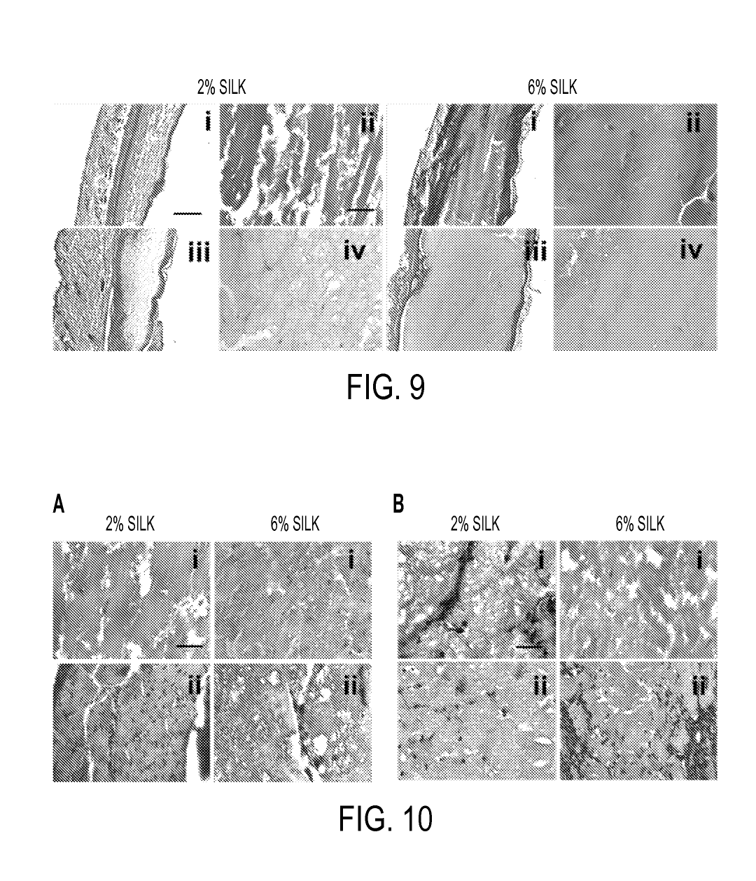

Since the therapeutic effects of PG are supposed to occur immediately, due to a fast release of growth factors in the site of injection, some animals injected with PG were sacrificed after 12 hrs and compared to PG mixed with silk. At 12 hrs following injection, the histological analysis, both H&E (FIG. 5Ai, ii) and Masson's Thricrome (FIG. 8) revealed the presence of the platelet gel with entrapped aggregated platelets. The immunohistochemistry analysis revealed that vascularization was stimulated, due to the presence of VE-cadherin and CD31 positive cells (FIG. 6Ai, Ci). At this initial time point, 2% and 6% silk-PG were the same size, and were much larger than PG alone in terms of both gross and histological analysis (FIG. 5Bi,ii). The 2% Silk-PG was more diffuse in eosin staining suggesting a softer network as compared to 6% Silk-PG, and neither contained CD31 positive cells within the gel bulk at this early time point, although positive staining was observed around the periphery (FIG. 6 B,D i). After 2 weeks from injection, the PG was still present, although diminished in size (FIG. 5Aiii, iv; 5Biii, iv), with new form vessels as revealed by the presence of VE-cadherin positive cell aggregates (FIG. 6Aii). Regarding the silk constructs, we observed after 2 weeks a significant decrease in size of 2% injected silk gels, with or without PG (FIG. 9). Only when mixed with PG, the 2% silk constructs started to be infiltrated with CD31 and VE-cadherin positive cells at 2 weeks, which progressively increased over 1 month. (FIG. 5Biii,iv; FIG. 6 B,D ii; FIG. 10). VE-cadherin positive staining was co-localized with the appearance of luminal structures characteristic of a capillary vascular phenotype. On the contrary, after 1 month the 2% silk construct without PG showed infiltration of only sparse hemotoxylin-positive infiltrating cells, presumably fibroblasts and inflammatory macrophages (FIG. 5 By, vi). Meanwhile, the overall dimensions of 6% silk constructs, both with or without PG, was relatively stable over the same 1-month time frame. Histologically, these groups showed only peripheral infiltration of cells and development of vessel-like structures at 2 weeks of observation, which showed only a small increase at 1 month (FIG. 6 Biii, Diii).

### Discussion

Autologous PG is widely used in regenerative medicine, such as in the acceleration of bone repair through the release of GFs from activated platelets (Simonpieri et al., Curr. Pharm. Biotechnol. 2012, 13, 1231). Several strategies have been recently proposed in order to improve the efficacy of PG injections, by optimizing growth factor release with different activators (Can et al., J. Thromb. Haemost. 2003, 1, 243) or by combining PG with bone allografts and bone marrow stromal cells (Dallari et al., J. Orthop. Res. 2006, 24, 877; Dallari et al., J. Bone Joint Surg. Am. 2007, 89, 2413). Another key aspect in the therapeutic efficacy of PGs is the spatial/temporal and bioavailability of the growth factors released in the site of injection. To address these limitations, we explored a new system of platelet growth factor delivery based on silk hydrogels. PGs, mixed with silk gels obtained by silk sonication slowed the release of GFs (VEGF, PDGF-AB and TGF-β1) from activated platelets as compared to PGs alone over 21 days. Upon solubilizing the PG-loaded silk gels using chaotropic agents, such as urea, we found that additional GFs were entrapped into the PG-Silk matrix. Together, these findings suggest that embedded GFs would be available in vivo by degradation-mediated release from the silk gel and would offer a mechanism by which silk could extend the therapeutic effects of PG-derived GFs.

The charge interactions of compounds with silk are known to result in different release profiles (Wang et al., Biomaterials 2010, 31, 1025; Lammel et al., Biomaterials 2010, 31, 4583). By modifying the net charge of silk with silk ionomers, silk-poly-L-lysine and silk-poly-L-glutamate (Calabrese et al. Biomaterials 2012, 33, 7375), we demonstrated that one of the mechanisms underlying silk-growth factor associations involves electrostatic interactions. Moreover, by using silk ionomers, the delivery of platelet growth factors can be temporally modulated by both electrostatic interactions and the swelling properties of silk. This mechanism is consistent with a model proposed by Guziewicz et al. in which increased hydrophobicity of the high density β-sheets decreases swelling and antibody release (Guziewicz et al., Biomaterials 2011, 32, 2642; Guiziewicz et al., Biomaterials 2013).

GF interactions with silk did not affect their biological activity, as they effectively promoted HUVEC proliferation via the canonical extracellular signal-regulated protein kinase activation pathway (pERK). As VEGF is one of the main GFs involved in HUVEC proliferation, by depleting VEGF from PG-Silk supernatant through a neutralizing antibody, we demonstrated that the released VEGF contributed, at least in part, in HUVEC proliferation through pERK pathway. Due to the incomplete attenuation of cell proliferation by blocking only the VEGF-specific pathway, it is likely that other factors active in our PG-Silk system (FGF, IGF, etc) could have contributed to our increases in HUVEC proliferation. We have also demonstrated that human mesenchymal cells respond similarly to hUVECS when PG-Silk is used to supplement growth media formulations (data not shown), suggesting that the bioactivity of the PG-Silk system is not completely VEGF-specific. All together, these findings demonstrate that silk is able to preserve the biological effect of platelet growth factors, which could potentially result in an in vivo therapeutic improvement of vascularization as well as bone formation due to the putative function of these well-characterized growth factors.

Another advantage conferred by silk to platelet GFs was improved stability. rVEGF was stable over time when re-suspended in silk solution; however, when re-suspended in PBS, rVEGF was quickly degraded. We also demonstrated that silk can stabilize TGF-β1 released from PG-Silk, as we observed significantly higher values than in PBS. Moreover, we showed that silk improves TGF-β1 stability over time, independent of storage temperature, as compared to plasma diluent. The mechanism for this enhanced stability must still be elucidated. Recently the stabilizing effect of silk on vaccines, antibiotics, and labile enzymes has been reported (Zhang et al., PNAS 2012, 109, 11981; Lu et al., Biomacromolecules 2009, 10, 1032; Pritchard et al., Biopolymers 2012, 97, 479). Here we reported for the first time the stabilizing effect exerted by silk in solution with respect to recombinant growth factors. We hypothesize that this enhanced liquid stability is conferred by silk's ability to reduce molecular mobility during storage and in turn, prevent protein unfolding and aggregation. It is therefore expected that in solution phase, before the gelation event has finalized, or during silk gel degradation, the stabilizing effect can preserve the initial amount of available GFs.

In addition to the biologic activity of the PG-Silk system, we also observed important mechanical benefits of silk to the PG. In particular, rheological data showed that not only the presence of a gel-forming silk protein did not interrupt the normal kinetics of PG formation, but the silk acted as an additional reinforcing agent to bulk up the gel construct in a manner dependent on the initial silk concentration. Interestingly, we observed that PG-laden silk gels were stiffer than their PPP-laden counterparts, suggesting that a fibrin-based PG was able to form first before the physical crosslinks of the silk gel formed and that the two acted in a synergistic manner. These behaviors are typical of sequential interpenetrating polymer networks, compatibility previously observed with other silk-and-protein hybrid gel systems (Gil et al., Macromol. Biosci. 2005, 5, 702; Gil et al., Mater. Sci. Eng. C 2007, 27, 426). The exact nature of the interaction between silk and the fibrin component of the PG network requires further study. Nonetheless, since demonstrating that the silk content can be increased to high protein loading in order to drastically enhance the mechanical properties (Kluge et al., J. Mech. Behay. Biomed. Mater. 2010, 3, 278; Wang et al., Biomaterials 2008, 29, 1054), we anticipate that final PG-Silk construct mechanical properties can be tuned in order to meet mechanical benchmarks established by native tissues, ranging from soft calcaneous fat tissue (Miller-Young et al, J. Biomech. 2002, 35, 1523) to stiff fibrocartilage (Johns et al., Proc. Inst. Mech. Eng. [H]. 2007, 221, 509), ˜8.5 kPa to ˜500-1000 kPa, respectively.

The degradation of injected platelet gels in some animal models (Cheng et al., J. Am. Coll. Cardiol. 2012, 59, 256) has been reported to occur in about 14 days, presumably by fibrinolysis. However, dependent on the silk concentration and formulation, injected silk gels demonstrate measurable degradation in several weeks (Zhang et al. Biomaterials 2011; Etienne et al., 2009, 80, 1852; Diab et al., J. Mech. Behay. Biomed. Mater. 2012, 11, 123). Our results provide further confirmation of the rapid degradation of PGs alone, in addition to the retention of silk gel for weeks following injection. While the rate of silk degradation is inversely proportional to concentration, the addition of PG had a normalizing effect, with both 2% and 6% degrading at the same rate. The increased stability and retention of the PG-silk constructs may enhance growth factor stability and subsequent cell infiltration, even with minimal silk content. Immmunohistochemistry confirmed the development of vascularized networks in all PG-silk constructs as the majority of infiltrated cells were both CD31 and VE-cadherin positive. These experiments demonstrate that by modulating the concentration of silk, the degradation rate of silk gels and in turn, the release of functional platelet growth factors, are modulated.

The inventors have identified several potential clinical applications for platelet gels based on current literature, and they are revisited here in terms of the mechanical properties of the implicated tissues. As shown in Table 2, unconfined compressive modulus values for these tissues range from ˜5 kPa to ˜1-4 GPa for calcaneous fat (soft tissue) subchondral bone (stiff tissue), respectively. PG-Silk compositions disclosed herein, likewise, can cover a modulus range of 3 orders of magnitude to specifically match native tissue compressive properties. Exemplary candidate formulations for these applications are indicated below.

TABLE 2
Compressive Properties of Representative
Human Tissues and Silk Gel Formulations

| | | | | | Final |
|---|---|---|---|---|---|
| | | | | | Silk |
| | Clinical | Modulus | | | w/v % |
| Tissue | Appli- | (Quasi- | | | Re- |
| Type | cation | Static) | | Authors, Citation | quired |
| Calcaneal | Diabetic | ~8.5 | kPa | Miller-Young, et al | 2% |
| fat pad | Foot | | | J. Biomech. 2002, | |
| | Ulcers | | | 35, 1523-1531 | |
| Nucleus | Inject IVD | 5.4 | kPa | Nerurkar, et al | 2% |
| Pulposus | Repair | | | J. Biomech. 2010, | |
| | | | | 43, 1017-1030 | |
| Passive | Soft | 1-4 | kPa | Van Loocke, et al | 2% |
| Muscle | Tissue | | | J. Biomech. 2006, | |
| | Filler | | | 39, 2999-3009 | |
| Supraspi- | Rotator | 24-90 | kPa | Johns et al., Proc. | 3-4% |
| natus | Cuff | | | Inst. Mech. Eng. H | |
| Tendon | Repair | | | 2007, 221, 509-526; | |
| | | | | Chahal et al., | |
| | | | | Arthroscopy 2012, | |
| | | | | 28, 1718-1727 | |
| Temporo- | Oral and | 211-514 | kPa | Johns et al., Proc. | 5% |
| mandibular | Maxillo- | | | Inst. Mech. Eng. H | |
| Joint | facial | | | 2007, 221, 509-526; | |
| | Recon- | | | Chin et al., J. Oral | |
| | struction | | | Maxillofac. Surg. | |
| | | | | 1996, 54, 315-318 | |
| Articular | Focal | ~1.5 | MPa | Armstrong et al, J. | 6-7% |
| Cartilage | Defect | | | Biomech. Eng. | |
| | Repair | | | 1984, 106, 165-173 | |

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A composition comprising silk fibroin and unlysed platelets.

2. The composition of claim 1, wherein the platelets are activated platelets.

3. The composition of claim 1 or 2, wherein the composition comprises platelet rich plasma.

4. The composition of any of claims 1-3, wherein the composition further comprises an active agent

5. The composition of claim 4, wherein the active agent is a coagulation agent.

6. The composition of claim 5, wherein the coagulation agent is thrombin.

7. The composition of any of claims 1-6 wherein the composition comprises from about 1% (w/v) to about 50% (w/v) silk fibroin.

8. The composition of any of claims 1-7, wherein the silk fibroin is substantially free of sericin.

9. The composition of any of claims 1-8, wherein the silk fibroin is obtained by boiling cocoons for from about 5 to about 120 minutes.

10. The composition of any of claims 1-9, wherein composition comprises silk fibroin modified with one or positively charged molecules.

11. The composition of claim 10, wherein ratio of ratio of modified to unmodified silk fibroin in the composition ranges from about 1000:1 (w/w) to about 1:1000 (w/w).

12. The composition of any of claims 1-11, wherein the composition comprises about 10³-10⁶ platelets/μL.

13. The composition of any of claims 1-12, wherein the composition further comprises an additive.

14. The composition of claim 13, wherein molar ratio of silk fibroin to additive in the composition is at least 10:1.

15. The composition of claim 13 or 14, wherein the additive is a surfactant.

16. The composition of claim 15, wherein the surfactant is selected from the group consisting of polysorbates, cationic molecules or polymers, and any combinations thereof.

17. The composition of claim 15 or 16, wherein the additive is polysorbate 80 (TWEEN® 80) or poly-lysine.

18. The composition of any of claims 1-17, wherein the composition further comprises a therapeutic agent.

19. The composition of any of claims 1-18, wherein the composition further comprises a wound healing agent.

20. The composition of any of claims 1-19, wherein the composition is a gel, hydrogel, foam, sponge, solution, fiber, tube, film, or non-woven mat.

21. The composition of any of claims 1-20, wherein the composition is adapted for injection or for topical application.

22. The composition of any of claims 1-21, wherein the composition further comprises a pharmaceutically acceptable excipient or carrier.

23. A composition for controlled release of a growth factor, the composition comprising silk fibroin and unlysed platelets.

24. An implant composition comprising a composition of any of claims 1-22.

25. A method of wound healing or wound closure, soft tissue repair or augmentation or reconstruction, hard tissue (musculoskeletal) repair or augmentation or reconstruction, filling a tissue located at or near a prosthetic implant, reconstructive oral and maxillofacial surgery, Achilles tendon repair, anterior cruciate ligament repair, augmenting bone implants, augmenting dental implants, bone and dental reconstruction, focal cartilage repair and microfracture augmentation, diabetic foot ulcers, cosmetic surgery and medicine, hair loss treatment, or wrinkle treatment, the method comprising administering a composition of any of claims 1-22 to the subject in need thereof.

26. A method for tuning stiffness of a hydrogel made through sonication, the method comprising varying one or more of the following:
(i) amount of silk in the hydrogel;
(ii) amount of platelets (e.g., platelet gel) in the hydrogel;
(iii) ratio of amount of silk to platelets (e.g., platelet gel) in the hydrogel;
(iv) silk fibroin concentration of the solution used to make the hydrogel;
(v) molecular weight of the silk in the hydrogel;
(vi) silk degumming time, e.g., cocoon boiling time;
(vii) amount of additive in the hydrogel;
(viii) net negative charge of silk in the hydrogel;
(ix) conformation of silk in the hydrogel; and
(x) any combination of (i)-(ix).

27. A method for altering the ionic and/or electrostatic properties of a hydrogel, the method comprising varying the net charge of silk fibroin in the hydrogel, wherein the net charge is varied by (i) modifying the silk fibroin with one or more negatively charged molecules; (ii) varying amount of modified silk fibroin; (iii) varying the ratio of modified silk fibroin to unmodified silk fibroin; or (iv) any combination of (i)-(iii).

28. A method of controlling degradation of an implant and therefore the growth factor delivery, the method comprising varying one or more of the following:
(i) amount of silk in the implant;
(ii) amount of platelets (e.g., platelet gel) in the implant;
(iii) ratio of amount of silk to platelets (e.g., platelet gel) in the implant;
(iv) silk fibroin concentration of the solution used to make the implant;
(v) molecular weight of the silk in the implant;
(vi) silk degumming time, e.g., cocoon boiling time;
(vii) amount of additive in the implant;
(viii) net negative charge of silk fibroin in the implant;
(ix) conformation of silk in the hydrogel; and
(x) any combination of (i)-(ix).

29. A method of controlling release of growth factors from an implant, the method comprising varying the net charge of the implant.

30. A composition comprising silk fibroin and growth factors derived by activated platelets, wherein the composition is substantially free of platelets.

31. A composition of any claims 1-7, wherein the silk fibroin is concentrated.

32. A composition of any claims 1-22, wherein the silk fibroin is entirely free or essentially free of organic solvents.

33. A composition of any claims 1-22, wherein the composition is sterilized.

34. A composition of any claims 1-22, wherein the composition is lyophilized for storage and recovery or the delayed degradation of encapsulated materials upon implantation.

35. A composition of any claims where the material gives solution stability.

36. A double-network hydrogel with a first network and a second network, wherein said first network comprises a platelet gel and said second network comprises a silk fibroin gel.

37. The hydrogel of claim 36, wherein said first network is semi-interpenetrated with said second network.
